(19) United States
(12) Patent Application Publication
(10) Pub. No.: US 2003/0108949 A1
(43) Pub. Date: Jun. 12, 2003

(54) Filtration-based microarray chip

(76) Inventors: Gang Bao, Mableton, GA (US); Yangqing Xu, Atlanta, GA (US)

Correspondence Address: William L. Warren, SUTHERLAND ASBILL & BRENNAN LLP, 999 Peachtree Street, NE, Atlanta, GA, 30309-3996, US

(21) Appl. No.: 10/189,923

(22) Filed: Jul. 3, 2002

Related U.S. Application Data
(60) Provisional application No. 60/303,278, filed on Jul. 3, 2001.

(51) Int. Cl. G01N 33/53; G01N 33/542; C12M 1/34

(52) U.S. Cl. 435/7.1; 435/7.9; 435/287.2

(57) ABSTRACT

Filtration-based microarray chips are provided. The microarray chips comprise a random oriented microporous filtration substrate of charged cellulose esters, such as nitrocellulose, and a plurality of different analyte-specific capture molecules attached to the substrate in a microarray. The capture molecules can be proteins, antigens, monoclonal or polyclonal antibodies, or apatomers. In particular, apparati and methods for the detection of a subject analyte comprising multiple stacked filtration-based microarray chips are also provided. The filtration-based microarray chips permits a sensitive, specific, fast and high throughput assay that can be used to detect analytes associated with a disease.

### CROSS REFERENCE TO RELATED APPLICATIONS

The present invention claims the priority benefit of U.S. Provisional Patent Application Serial No. 60/303,278 filed Jul. 3, 2001, the entire contents of which are hereby incorporated by reference.

### FIELD OF THE INVENTION

This invention relates generally to compositions and methods for the detection of biological analytes in a sample. More specifically, the present invention relates to assays using a microarray of capture molecules for high throughput detection of target analytes, such as those in a sample associated with disease.

### BACKGROUND OF THE INVENTION

As an emerging field in life sciences, proteomics is based upon, and developed beyond, genomics [2][42]. The word proteome describes the total protein output encoded by a genome. Proteomics is “the use of quantitative protein-level measurements of gene expression to characterize biological processes (e.g. disease processes and drug effects) and decipher the mechanisms of gene expression control” [2]. The study of genomics has provided knowledge of gene expression and regulation affected by processes such as drug treatments or disease states. The development of new technologies including serial analysis of gene expression (SAGE) [56] and DNA chips[44], which are now commercially available, has greatly advanced genomic studies. In the case of DNA chips, such as GeneChip® developed by Affymetrix, changes in the expression levels of a predetermined set of genes can be examined. In this method, oligonucleotide probes corresponding to a portion of different genes are selected according to the specific application interest and immobilized onto a solid surface to form an array. mRNAs from the sample of interest are amplified and fluorescently tagged, then added to this probe array, and allowed to hybridize to the corresponding oligonucleotide probes. With this approach, changes in mRNA concentration levels of >2fold can be detected. These DNA microarrays can be utilized not only to examine the changes in gene expression but also to generate a database of patterns of gene expression or gene expression changes which can then be associated with a certain phenotype or physiological state. DNA chips, however, cannot be used to quantify protein expression levels, which are usually different from gene expression levels due to post-transcriptional control and post-translational modifications [3][17].

Proteins play central roles in establishing the biological phenotype of organisms; therefore, proteins are more indicative of functionality whereas the mRNA transcripts are indicative of genetic information. Although mRNA expression levels may seem to be an indication of protein expression, studies using different and independent approaches have shown that due to alternative splicing and other regulation mechanism, there does not exist a strong correlation between mRNA and protein abundance [3]. Further, genomic studies cannot provide information on post-translational modifications of proteins, which are of great importance to health and disease.

At the present, a great deal of proteomic studies rely on 2-D polyacrylamide gel electrophoresis (2-D PAGE) [13] where proteins from a sample of interest such as cell lysate or tissue homogenate are separated by their molecular weight and isoelectric points. Upon separation of the proteins, the resulting gel will be stained and analyzed by computer software to provide quantitative results on fold-induction or repression of protein expression between samples, such as diseased and non-diseased states or control and drug-treated samples. Historically, methods such as amino acid analysis and Edman sequencing have been employed to obtain protein identities from 2-D PAGE systems. However, it has been well recognized that 2-D PAGE is time-consuming and its results are difficult to interpret. Recently, mass spectroscopy has been utilized more extensively for high-throughput identification of unknown protein samples. However, mass spectroscopy requires specific and expensive instrument, and this may hinder its clinical applications. In addition to 2-D PAGE and mass spectroscopy, immunoassays such as ELISA (enzyme linked immunosorbent assay), FIA (Fluorescence immunoassay) and RIA (Radio immunoassay) have also been developed for detecting certain protein targets [4][12]. These methods either lack the desired specificity (2-D PAGE) or are limited by the low throughput (FIA, ELISA and RIA).

The diffusion-limited hybridization in solid-phase assays has received extensive studies since the early 1980s. To expedite the reaction kinetics in the conventional ELISA (Enzyme Linked Immuno Sorbent Assay), the ELIFA (Enzyme Linked Immuno Filtration Assay) was invented (see http://www.piercenet.com/files/ACF15B.pdf) [22][34][37][38]. However, ELIFA is a low through-put assay designed to have one type of capture molecule per well, thereby limited by the number of wells used. Further, the amount of sample required for the current design of ELIFA is much higher than that for protein microarray chips.

The recently developed protein chips, or protein microarrays have the potential to overcome the drawbacks of conventional methods. This microarray technology is a novel and powerful tool for high throughput assaying of proteome [23], including protein-protein interactions [27], enzyme activity [60] and protein detection [23]. Most of the current protein chips are based on the reactions between the capture proteins immobilized on a surface and the analyte proteins in the sample solution. Briefly, a series of capture proteins such as antibodies is first spotted onto a solid surface to form a microarray. A small volume of fluorescently labeled protein sample is then applied to the surface. After a sufficient period of incubation with shaking, sample proteins bind to their matching capture proteins. The binding events can be visualized by spatially resolved fluorescent signals, the intensity of which reflects the quantity of the analyte in the sample and the binding affinity of the analyte to the capture protein. Since a protein microarray may contain many types of capture proteins; it can simultaneously detect multiple analytes or study protein interactions.

Macbeath and Schreiber [27] have demonstrated for the first time the potential of protein chips for high throughput studies of protein-protein interactions on a solid substrate. Briefly, in a specific assay, 10,800 spots were printed for protein G (10,799 spots) and FRB (1 spot) on one piece of coverslide. The slide was probed with a mixture of BODIPY-FL-IgG, Cy5-FKBP12 and rapamycin. After incubation and removing unhybridized analytes, it was observed that the spot with FRB showed elevated signal intensity in the Cy5 channel, which proved that FKBP12 was captured by FRB. In another assay, three different proteins were spotted on a coverslide, including Anti-DIG, Streptavidin, and FKBP12. Alexa488-BAS-DIG, Cy5-Biotin-BSA and Cy3-BSA-AP1497 were incubated individually with the microarray, and only the spots with the corresponding capture molecule showed fluorescence. Additionally, all three kinds of target molecules were incubated with the microarray, and all spots showed fluorescence signals. This proved the principle that microarray can specifically detect the existence of multiple analytes.

A number of recent studies have shown the great potential of this enabling technology [23][27][57][60]. However, most of the current protein chip designs have followed the same approach as in the development of DNA microarrays, i.e., printing spots with capture molecules on a glass coverslide and performing incubation and shaking. This approach can be very problematic for proteomic studies due to the sever diffusion limit in protein hybridization and the less-ideal substrate properties for binding capture molecules. Specifically, for microarrays printed on a solid surface, the reactions and hybridization between analytes and the immobilized capture molecules rely on shaking, which are limited by the slow diffusion of analytes across the boundary layer at the liquid-solid interface, i.e., the reaction is diffusion limited [51]. Since proteins cannot be amplified, this limit is far more severe for protein chips than for DNA chips.

Given the fundamental differences between proteins and nucleotides, glass coverslide is not optimal for protein immobilization. In contrast to the linear structure of oligonucleotides, proteins have complex three-dimensional conformations, and any major deviation from the native conformation may alter the normal functions of the protein molecule [54][55]. Therefore, one major concern for conventional protein chips is whether capture molecules (e.g., antibodies) will maintain their normal functions upon binding to the hydrophobic coverslide surface. Another major issue is that DNA-DNA and protein-protein interactions may have very different affinities and specificities. DNA-DNA interactions are through long-range base pairing, and they have much lower disassociation rates, much higher affinity and much better specificity than protein-protein interactions, which rely on local conformational matches. Thus, protein microarrays require either high concentration of analytes or high surface concentration of the capture molecules. Unlike DNA, protein samples cannot be amplified, so the only practical approach is to increase the amount of capture molecules immobilized on the surface. Therefore, the substrates for protein chips should have high protein-binding capacity. However, a high surface concentration of capture molecules inevitably leads to more severe diffusion limit and slower hybridization kinetics.

To overcome the diffusion limit, different approaches have been developed to accelerate the reaction. For example, Nanogene developed DNA microarrays using electrophoresis techniques. The hybridization is accelerated by an electric field that guides the motion of target toward immobilized probes. However, due to the more complicated isoelectric properties of proteins, this approach is not directly applicable to protein chips since the protein molecules may move in different directions and with different rates under an applied electric field. More recently, a flow-thru DNA chip has been developed by Genelogics [8]. In this approach, DNA probes are immobilized on a chemically modified porous silicon wafer. Fluorescently labeled DNA targets then flow through the silicon wafer. This approach significantly accelerates the reaction kinetics since the diffusion limit is reduced. In addition, since porous silicon wafer has a large surface area and can bind more probes, a better signal intensity was obtained compared with the conventional DNA chip. A similar design was adopted by MetriGenix to develop a flow-thru 4D chip platform for high-throughput DNA sequencing. However, silicone wafers require the modification of the surface chemistry in order to bind proteins, and the resulting coating is usually unstable [49].

### SUMMARY OF THE INVENTION

The present invention provides compositions and methods for the detection of a subject analyte comprising a filtration-based microarray chip comprising, one or more planar filtration substrates comprising charged cellulose esters with randomly oriented micropores, and a plurality of different analyte-specific capture molecules attached to the substrate in a microarray, wherein the filtration-based microarray chip permits a fluid solution to flow therethrough and at least a portion of analytes to be captured thereto. In certain embodiments of the microarray chip, the charged cellulose esters are cellulose nitrate, or mixtures of cellulose nitrate and cellulose actetate.

Some preferred embodiments of the present invention provide that the analyte is a protein, such as an antibody or an antigen, obtained from a sample of cell lysate, or bodily fluid. Some preferred embodiments of the present invention provide that the analyte-is a small molecule. Some preferred embodiments of the present invention provide that up to ten or more different analyte-specific capture molecules are proteins, antibodies, or nucleic acids.

The present invention provides an apparatus for analyte detection comprising a plurality of filtration-based microarray chips as described herein aligned with planar aspects in parallel such that a solution flows through the plurality of chips. This apparatus can further comprise a holder for the chips and a means for washing a solution of analytes repeatedly through the plurality of chips. An apparatus is provided by the present invention with two or more microarray chips stacked together, and wherein the analyte to be analyzed is filtrated through the entire stack.

The invention provides methods of detecting a subject analyte, comprising combining the micrarray chip of the invention with a sample suspected of containing the subject analyte, and detecting the capture of the analyte on the substrate to determine the presence of the subject protein in the sample. The invention provides in certain embodiments that the detection of the subject analyte indicates the presence of a marker of a disease in the sample.

Accordingly, it is an object of the present invention to provide improved compositions and methods of use for more sensitive, specific or rapid analyte detection.

It is a further object of the present invention to provide improved assays for the high throughput detection of multiple analytes in a solution.

It is a further object of the present invention to provide improved assays for the detection of analytes associated with a disease or condition for diagnosis.

These and other objects, features and advantages of the present invention will become apparent after a review of the following detailed description of the disclosed embodiments and the appended claims.

### BRIEF DESCRIPTION OF THE FIGURES

FIGS. 1a-1c show the electron microscopy image of silicone wafer and Nitrocellulose. In FIG. 1a a 150-micron thick wafer piece is shown, and the cross-sections of the microchannels is shown in FIG. 1b. The channel diameter is about 1.5 micron (Ref. 58). FIG. 1c shows the cross-section of Nitrocellulose filter (mixed ester). Scale bar is 10 micron.

FIGS. 2a and 2b illustrate the multi-chip stacking-hybridization system. Hybridization on conventional coverslide and through multi-chip stacking of filter-based chips is shown in (2a) and (2b), respectively.

FIGS. 3a and 3b show two embodiments of the apparatus of the invention. FIG. 3(a) illustrates the system comprising a filtration device, a syringe pump that drives multiple syringes, a chip holder, and the associated tubing. FIG. 3(b) shows in more detail a design of the chip holder.

FIG. 4 gives an example of the pattern of microarray for hybridization studies. For each test case in this example (e.g., an antibody-antigen pair) three spots are used. The first five rows are for different antibody-antigen pairs; the last row serves as the control (standard).

FIGS. 5a and 5b show the effects of detergent and washing methods on the binding of labeled proteins to nitrocellulose.

FIG. 6 shows the effect of detergent on the binding of unlabeled proteins to the nitrocellulose membrane.

FIGS. 7a and 7b give the results of hybridization using (7a) nitrocellulose chip and (7b) glass coverslide chip.

FIG. 8 demonstrates the difference in hybridization kinetics of filtration and shaking assays for CEA binding to 1 mg/ml Anti-CEA spots.

FIGS. 9a-9d show the images of protein chips after 15 minutes (9a filtration assay & 9b shaking assay) and 45 minutes of hybridization (9c filtration assay & 9d shaking assay).

FIG. 10 displays the normalized signal intensity of both filtration and shaking assays after 60 minutes of hybridization.

FIGS. 11a-11c show the detection of low concentration of proteins after 30 minutes of hybridization. (11a) Filtration assay, with analyte concentration of 0.064 ng/ml. (11b) Shaking assay, with analyte concentration of 0.064 ng/ml. (11c) Shaking assay, with analyte concentration of 1.6 ng/ml.

FIGS. 12a-12d show the dynamic ranges of the filtration-based protein microarrays for capturing (12a) HSA, at AHSA concentration of 1.0 mg/ml, (12b) CEA, at ACEA concentration of 1.0 mg/ml, (12c) MGG, at GAM concentration of 1.0 mg/ml, and (12d) Neutravidin, at Ca-Biotin concentration of 1.0 mg/ml.

FIG. 13 compares the dynamic range of filtration assay and shaking assay for CEA, with ACEA concentration of 1.0 mg/ml.

FIG. 14 illustrates how the dynamic range varies with surface concentration for filtration assay.

FIG. 15 shows the comparison of backgrounds in filtration assay and shaking assay.

FIGS. 16a-16c indicate that filtration-based protein chips can reduce the nonspecific binding that may occur after hybridization for a long time. First row, AHSA, ACEA; Second row, GAM, PG. (16 Filtration hybridization, 60 minutes; (16 shaking hybridization, 60 minutes; (16 shaking hybridization, overnight.

FIG. 17 shows that the filtration-hybridization of AHSA to spots of HSA was uniformly distributed along the diameter of a 13-mm large chip. Procedure: HSA-Alexa546 was spotted along the diameter of the filter. Then the spotted chip was hybridized with Anti-HSA-Alexa647. After hybridization, the chip was imaged, and the spot intensities in both fluorescence channels were quantified. The ratio between A647 and A546 is plotted in FIG. 17.

FIG. 18 shows that the filtration-based hybridization through a stack of 8 chips gives very uniform results. The top two rows (Chips 1-8) are the results of the stacked-filtration assay while the bottom row (Chips 9-12) shows the results of shaking hybridization, with an average sample volume per chip of 60 ul.

FIGS. 19a-19b demonstrate the consistency in fluorescence intensity of the multi-chip stacking hybridization assay. FIG. 19a shows an embodiment where 6 chips are stacked.

FIG. 19b shows an embodiment where 8 chips are stacked.

FIGS. 20a-20d show the results of selective detection of HSA by a single positive chip (Top) stacked between negative chips (Bottom). (a) Positive chip, visualized in the Alexa-647 signal channel. The frame highlights the capture of HSA-A647 by the AHSA spots. (b) Positive chip, visualized in the Alexa-488 signal channel. (c) Negative chip 1, visualized in the Alexa-647 signal channel. (d) Negative chip 2, visualized in the Alexa-488 signal channel.

FIG. 21 shows the comparison of the detection of streptavidin-PB1L (2 μg/ml) and streptavidin-A647 conjugates (20 ng/ml) by immobilized biotin-BSA on nitrocellulose surface. The molar concentrations of streptavidin are the same for all the samples. The results are based on: (a) filtration assay, using streptavidin-PB1L, (b) filtration assay, using streptavidin-A647, (c) shaking assay, using streptavidin-PB1L, (d) shaking assay, using streptavidin-A647. The chart on the right shows the quantification of spot intensity in different tests.

FIGS. 22a and 22b demonstrate the uniformity of the results using the multi-chip stacking-hybridization using PB1L. FIG. 22a is an image of the top chip and FIG. 22b is an image of the bottom chip in a stacked filtration system.

FIG. 23 shows that the sandwich assay gives a much better contrast than the direct conjugation assay with labeled proteins, and that filtration assay works better than shaking assay.

FIG. 24 reveals that that filtration-based sandwich assay can detect 5 ng/ml of CEA concentration elevation in a healthy individual's blood.

FIG. 25 demonstrates the detection of CEA in pancreatic cancer patient's plasma. Negative controls were collected from two healthy donors. Two chips were hybridized with each negative control, while three tests were repeated for cancer plasma. Estimated by unpaired t-test, the results yielded from all the spots showed that the difference between this cancer sample and healthy donors' plasma is statistically significant.

FIG. 26 displays the pattern of microarray for apatomer hybridization studies.

FIG. 27 shows the specificity of the aptamer chip by comparing the results of (a) 10 ng/ml Thrombin-A647, filtration assay, 10 min and (b) 1 μg/ml HAS-A647, filtration assay, 10 min.

FIG. 28 is a comparison of the kinetic rate for filtration and shaking assays of apatomer chips

FIG. 29 shows a comparison between the results obtained from filtration assay (left panel) and shaking assay (right panel) of the aptamer chip after 10 minutes of hybridization.

FIG. 30 shows the dynamic range of the aptamer chip in detecting thrombin.

### DETAILED DESCRIPTION OF THE INVENTION

The present invention may be understood more readily by reference to the following detailed description of the preferred embodiments of the invention and the Examples included therein. Before the present compounds, compositions, and methods are disclosed and described, it is to be understood that this invention is not limited to any specific substrates, specific antibody or other capture molecules, specific analytes, specific conditions, or specific assays or methods, etc., as such may, of course, vary, and the numerous modifications and variations therein will be apparent to those skilled in the art. It is also to be understood that the terminology used herein is for the purpose of describing particular embodiments only and is not intended to be limiting. As used in the specification and in the claims, “a” or “an” can mean one or more, depending upon the context in which it is used. Thus, for example, reference to “a capture protein” or “an analyte” can mean that one or frequently more than one can be utilized.

In accordance with the purpose(s) of this invention, as embodied and broadly described herein, this invention, in one aspect, provides a filtration-based microarray chip comprising, one or more planar filtration substrates comprising charged cellulose esters with randomly oriented micropores, and a plurality of different analyte-specific capture molecules attached to each substrate in a microarray, wherein the filtration-based microarray chip permits a fluid solution to flow therethrough and at least a portion of analytes to be captured thereto.

In certain embodiments of the microarray chip, the charged cellulose esters are cellulose nitrate, or mixtures of cellulose nitrate and cellulose actetate. In accordance with this embodiment of the present invention, an additional two-dimensional solid surface, such as a glass coverslide, is not incorporated as part of the substrate in order to permit the fluid solution of analytes to flow through the substrate.

Charged cellulose esters are well-known in the art. In a common manufacturing process for making cellulose nitrate, nitrate groups substitute the hydroxyl moieties on each sugar unit through treatment with nitric acid. In a common manufacturing process for making cellulose acetate, ester groups substitute the hydroxyl moieties on each sugar unit through treatment with acetate acid. Nitrocellulose generally refers to a mixture of cellulose esters: cellulose nitrate and cellulose acetate. In some embodiments, the nitrocellulose refers to a mixture of about 90% cellulose nitrate and about 10% cellulose acetate.

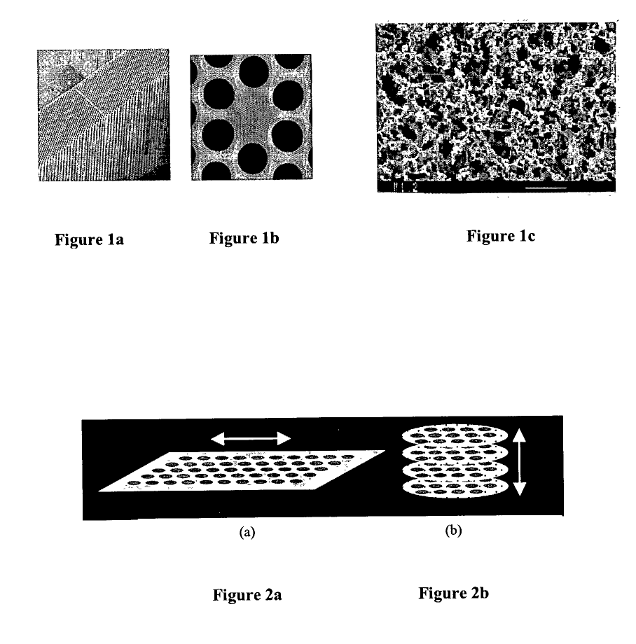

Dry nitrocellulose is readily soluble in organic solvents forming a lacquer. Evaporation of the solvents results in deposition of the polymer as a thin film. Pores can be introduced into the film to create a microporous membrane including a non-solvent, such as water, in the lacquer. Pore formation can result from the differential evaporation of the solvent and non-solvent. Thus, porosity and pore size can be controlled easily by the amount of non-solvent in the lacquer. The resulting film is a three-dimensional highly porous structure, such as that shown in cross-section in FIG. 1c.

Addition of a surfactant during the casting process allows deposition of molecules in aqueous buffers. The most commonly used surfactants are anionic detergents such as sodium dodecyl sulfacte (SDS) or Triton X100. Immobilization of analytes can typically be achieved through drying. Some preferred embodiments of the present invention provide that the filtration substrate micropores are between about 0.05 and 10 microns in diameter. Typically, the micropores can be between about 0.2 and 5 microns.

Optionally, the filtration substrate may comprise a combination of more than one form of chemical additives. The filtration substrate can have functionalities exposed on its surface that serve to enhance the surface conditions of a substrate or a coating on a substrate in any of a number of ways. For instance, exposed functionalities are typically useful in the binding or covalent immobilization of the proteins to the array. Alternatively, the filtration substrate may bear functional groups (such as polyethylene glycol (PEG)) which reduce the non-specific binding of molecules to the surface. Other exposed functionalities serve to tether the analyte-specific capture molecule to the surface of the substrate or the coating such as streptavidin for capture of biotinylated analytes. Particular functionalities of the filtration substrate may also be designed to enable certain detection techniques to be used with the surface. Alternatively, the filtration substrate may be modified to serve the purpose of preventing migration or inactivation of a capture molecule immobilized on a patch of the microarray.

Some preferred embodiments of the present invention provide that the analyte is a protein. The analyte protein can be, for example, an antibody. In preferred embodiments, the analyte is obtained from a cell lysate or a sample of bodily fluid. Therefore, the detection of an analyte known to be associated with a disease or condition can serve as a diagnostic or prognostic indicator of the presence of the disease or condition in the sample. It will be appreciated by those in the art that the analyte can be any molecule capable of being captured on the microarray chip as described herein. For example, the analyte can be a member of a library of chemically synthesized molecules that is a therapeutic drug candidate. The high throughput capability of the present invention permits the screening of a large number of analytes for interaction with the analyte-specific capture molecules on the microarray chip.

Some preferred embodiments of the present invention provide that ten or more different analyte-specific capture molecules are attached to the substrate in a microarray. In certain embodiments the analyte-specific capture molecules are antibodies, for example either monoclonal or polyclonal antibodies. The invention provides that the analyte-specific capture molecules can be any amino acid based molecules, or nucleic acid based molecules, or a combination of amino acid based and nucleic acid based molecules, either naturally occurring or recombinantly or synthetically produced by techniques well known in the art.

An “array” is an arrangement of capture molecules, particularly biological macromolecules (such as polypeptides or nucleic acids) in addressable locations on a substrate. A “microarray” is an array that is miniaturized so as to require minimal amount of capture molecules and sample for evaluation. Within an array, each arrayed molecule is addressable, in that its location can be reliably and consistently determined within the at least two dimensions of the array surface. Thus, in ordered arrays the location of each molecule sample is assigned to the sample at the time when it is spotted onto the array surface and usually a key is provided in order to correlate each location with the appropriate target. Often, ordered arrays are arranged in a symmetrical grid pattern, but samples could be arranged in other patterns (e.g., in radially distributed lines or ordered clusters).

The shape of the sample application of capture molecule or “spot” is immaterial to the invention. Thus, the microarray of capture molecules refers generally to a localized deposit of analyte-targeting polypeptide or oligonucleotide, and is not limited to a round or substantially round region. For instance, essentially square regions of polypeptide or oligonucleotide application can be used with arrays of this invention, as can be regions that are essentially rectangular (such as slot blot application), or triangular, oval, or irregular. The size (diameter of a circular area enclosing the entire spot therein) of the spot itself is immaterial to the invention, though it is usually between 0.1 mm to 0.5 mm. The shape of the array itself is also immaterial to the invention, though it is usually substantially flat and may be rectangular or square in general shape. The preparation of a microarray of the present invention refers to the arrangement of different groups of analyte-specific capture molecules in a spot-to-spot (edge-to-edge) spacing of between about 0.05 mm to 10 mm, or more preferably of between about 0.2 mm to 1 mm.

Arrayers, also named array spotters, useful in the present invention are currently available from multiple companies. According to the different spotting techniques, they can be classified into contact mode and non-contact mode (inkjet) arrayers [61]. Contact mode arrayers, including systems manufactured by Affymetrix, Amersham Pharmacia, BioRobotics, and GeneMachine, for example, use pen tips that dispense the sample when the tips touch the substrate. The non-contact mode arrayer, represented by BioChip arrayer manufactured by Packard Bioscience (now part of Perkin-Elmer Bioscience), employ piezo-controlled tips to dispense the pre-sucked sample at about 400 μm above the substrate [61].

The criterions of selecting a spotting system for a given application include the throughput required by the application, the speed of printing, the nature of the sample (Protein or DNA), the accuracy in controlling the sample volume, and the maximal spot intensity. In general, the tips used in contact mode arrayers are cheaper, and the simple dispensing process do not require delicate control for individual tips, so relatively more tips can be installed in the same arrayer. For example, 64 tips can be simultaneously installed in MicroGridII system from BioRobotics. Apparently, more tips can improve the speed of dispensing. In contrast, non-contact mode arrayers require individual pressure control and dispensing control system for each tip, and this increases the cost per tip and limits the throughput of the instrument. One will note that the number of source plates that an arrayer can take also influences the throughput, but it can be altered by the accessories equipped with the arrayer, such as a microplate autoloader; therefore, the details of different systems are not compared here and the appropriateness of each system will become apparent to one of skill in the art depending upon the circumstances.

In spite of the lower throughput, compared to contact mode systems in which the surface tension of the sample decides whether it can be dispensed onto the substrate, non-contact mode systems actively shoot the sample out and can therefore work with more viscous samples such as high concentration protein solutions. This technique also reduces the tip-to-tip variances in contact mode arrayers. The sonicating device in each individual tip of BioChip arrayer greatly reduces tip contamination. Also, the control of dispense volume is featured in non-contact mode arrayer while it cannot be realized by non-contact mode arrayers. Typically, the maximal spot intensity of arrays generated by contact mode arrayer can be as high as 64 spots/mm², while BioChip arrayer can spot 16 spots/mm².

An analyte may be shared by more than one analyte-specific capture molecule. For instance, a binding partner which is bound by a variety of polyclonal antibodies may bear a number of different epitopes. One capture agent may also bind to a multitude of binding partners (for instance, if the binding partners share the same epitope). “Conditions suitable for protein binding” means those conditions (in terms of salt concentration, pH, detergent, protein concentration, temperature, etc.) which allow for binding to occur between an analyte and its analyte-specific capture molecule in solution. Preferably, the conditions are not so lenient that a significant amount of non-specific binding occurs. However, a sandwich assay will improve the specificity significantly.

The term “normal physiological condition” means conditions that are typical inside a living organism or a cell. While it is recognized that some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. It will be recognized that the concentration of various salts depends on the organ, organism, cell, or cellular compartment used as a reference.

The analyte-specific capture molecules are capable of binding to at least one specific analyte of interest. As used herein, the terms “bind”, “hybridize” and “capture” are used synonymously to indicate an interaction between an analyte and an analyte-specific capture molecule on the microarray chip that can be detected either directly or indirectly by any method or technique, such as but not limited to those described herein. In some embodiments, it may be useful to detect binding under normal physiological conditions.

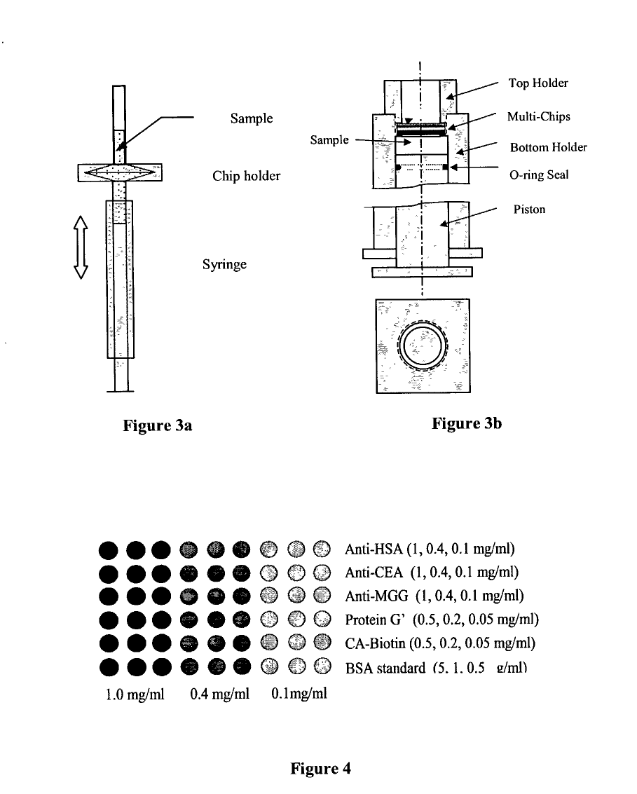

The present invention provides an apparatus for analyte detection comprising a plurality of filtration-based microarray chips as described herein aligned with planar aspects in parallel such that a solution flows through the plurality of chips. Microarray chips as described herein aligned with planar aspects in parallel such that a solution can flow through the plurality of chips is shown schematically in FIG. 2b. An exemplary apparatus is shown in FIGS. 3a and 3b, which can further comprise a holder for the chips and a pump means for washing a solution of analytes repeatedly through the plurality of chips. In one exemplary such embodiment, the means for washing the solution of analytes repeatedly through the plurality of microarray chips is a syringe in fluid communication with a chamber housing the chip holder and the aligned chips. One or more such syringes can be connected to an automatic pump to provide a predetermined and consistent rate of flowthrough of the sample across the chips. Preferably, the apparatus is maintained in a vertical position with an open top as shown for easy delivery of a sample and to avoid entrapment of air in the microarray chips during flowthrough pumping.

Therefore, an apparatus is provided by the present invention of 1, 2, 3, 4, 5, 6, 7, 8, 9, 10 or more microarray chips stacked together, and the analyte to be analyzed is filtrated through the entire stack. When the stacked chips contain different bound capture molecules, the stacked multichip system can selectively detect an analyte by using capture molecules attached, such as by well-known micro-printing techniques on any layer of the stack. For example, the invention contemplates a square filter with each side 22 mm in length. With a microarray of the present invention printed with the spot-to-spot spacing of 0.5 mm, then at least 1,600 spots can be arrayed on each chip of this example. If 10 chips are used to form a stack, then 16,000 spots can be simultaneously utilized for detection of analytes of interest.

The invention provides a method of detecting a subject analyte, comprising combining the micrarray chip of the invention with a sample suspected of containing the subject analyte, and detecting the capture of the analyte on the substrate to determine the presence of the subject protein in the sample. The invention provides in certain embodiments that the detection of the subject analyte indicates the presence of a disease marker in the sample. In certain embodiments, the analyte is obtained from a cell lysate or a sample of bodily fluid. A “body fluid” may be any liquid substance extracted, excreted, or secreted from an organism or tissue of an organism. The body fluid need not necessarily contain cells. Body fluids of relevance to the present invention include, but are not limited to, whole blood, serum, urine, plasma, cerebral spinal fluid, semen, tears, sinovial fluid, and amniotic fluid.

In certain embodiments, the detection of the analyte is achieved by labeling the analyte before capture. For example, the analyte can be labeled with a fluorescent dye. Alternatively, the invention provides for the detection of the analyte by a secondary labeling antibody after the analyte is hybridized to the capture molecule, such as in a modified ELISA assay. For example, the antibody can be labeled with a fluorescent dye, and the signal can be detected by a fluorescence imager or a microarray scanner. Alternatively, labeling can be achieved with any other known system including calorimetric detection, luminescence, chemiluminescence, or radioisotopes, for example.

In one embodiment, the method further comprises the intermediate step of washing the microarray to remove any unbound or nonspecifically bound components of the sample from the array before the detection step. In another embodiment, the method further comprises the additional step of further characterizing the particular analyte retained on at least one particular capture molecule, or portion of the microarray chip.

The invention further provides kits for the detection of a subject analytes comprising the microarray chips described herein, necessary reagents and instructions for practicing the methods of detection. Such alternative compositions, methods and kits therefor are described in more detail by way of the examples, and still others will be apparent to one of skill in the art in view of the present disclosure.

The analytes or the analyte-specific capture molecules of the present invention may be substantially isolated or alternatively unpurified. An “isolated” or “purified” substance is one that is substantially free of material with which is naturally associated, such as other cellular material, or culture medium when produced by recombinant techniques, or chemical precursors or other chemicals when chemically synthesized (see, Sambrook et al. 1989, Molecular Cloning: A Laboratory Manual. 2nd, ed., Cold Spring Harbor Laboratory, Cold Spring Harbor Laboratory Press, Cold Spring Harbor, N.Y.).

Other embodiments of the invention provide methods of analyzing proteins, particularly protein-molecule interactions and/or binding characteristics. Certain of these methods include obtaining more than one (a plurality) substantially pure protein specimen, placing a sample of each specimen in an addressable location on a microarray; and probing the array of specimens with a detectable probe molecule. Arrays for use in these methods can be macro- or microarray, or combinations thereof. Probe molecules used to assay arrays in these methods can be any molecule, for example a polypeptide, a ligand, an oligonucleotide, a fragment thereof, or mixtures thereof.

Other methods provided include methods of analyzing a plurality of binding characteristics of an array of polypeptide samples. In such methods, an array of polypeptide samples is probed at least twice, sequentially, with at least a first and a second (different) analyte or probe molecule. The array may be stripped of bound first probe prior to being assayed with the second probe. Binding patterns for the first and second probes can be detected and analyzed to determine which polypeptides each probe binds to, thereby revealing multiple binding characteristics of the array of polypeptide samples.

For microarray technique, fluorescent detection is commonly used. To reliably image the high-density arrays that consist of spots as small as 100 μm, high-sensitivity high-resolution fluorescence microarray scanners have been developed. Currently, the major companies that produce array scanners include Affymetrix, Amersham Pharmacia, Packard Bioscience, and Genomic solutions, for example (see, Ramdas L., Zhang W., What is happening inside your microarray scanner? Biophotonics 2002 March, p42-47). The light source can be either laser (more monochromatic, less cross-talk between channels) or white light source (more flexible for different excitation requirements). The detectors used are PMT (images are reconstructed from pixels that have be sequentially scanned) or CCD camera (images are integrated from all the pixels that are simultaneously imaged). Currently, most array scanners are based on the laser-PMT system, and their maximal resolutions are usually less than 5 μm. Sensitivity as high as the detection of one fluorescence molecule per μm² is obtained, but one should be aware that the sensitivity for an actual assay may be limited by other factors such as non-specific binding backgrounds. More features such as confocal microscopy imaging or dark field imaging can be added. However, confocal microscope based arrayers such as ScanArray (Packard Bioscience) may not be suitable for non-flat or thick substrates that require thicker focus plane because they fail to collect all the signals through the substrate. Dark-field arrayers reduce the absolute intensity of signals, but they are capable of improve the signal contrast on surfaces where scattering is high, such as nitrocellulose membrane. Overall, Dark-field scanners such as GeneTac LSIV (Genomic solutions) that has a depth of focus as long as +/−500 μm, and a resolution of 1 μm, may preferably image the nitrocellulose filtration-based chips than the ScanArray (Packard Bioscience, confocal based, 30 μm focus depth, and 5 μm resolution), which is currently used in developing the present examples. Alternatively, when the resolution and sensitivity is not critical, fluorescence imagers that conventionally used for gel and blot imaging can also be applied for microarray scanning. A FLA-3000 imager (Fuji) that was used in this research has a minimum 50 μm resolution but longer focal length, and it proved to be more accurate in quantify the amount of fluorescence on nitrocellulose filters.

Moreover, a wide range of detection methods is applicable to the methods of the invention. As desired, detection may be either quantitative or qualitative. The invention array can be interfaced with optical detection methods such as absorption in the visible or infrared range, chemoluminescence, and fluorescence (including lifetime, polarization, fluorescence correlation spectroscopy (FCS), and fluorescence-resonance energy transfer (FRET)). Furthermore, other modes of detection such as those based on optical waveguides surface plasmon resonance, surface charge sensors, and surface force sensors are compatible with many embodiments of the invention. Quartzcrystal microbalances and desorption processes provide still other alternative detection means suitable for at least some embodiments of the invention array. An example of an optical biosensor system compatible both with some arrays of the present invention and a variety of non-label detection principles include surface plasmon resonance, total internal reflection fluorescence (TIRF), Brewster Angle microscopy, optical waveguide lightniode spectroscopy (OWLS), surface charge measurements, and ellipsometry. Quantum dots are a particularly useful detection technique with the present invention.

Unless otherwise noted, technical terms are used according to conventional usage. Definitions of common terms in molecular biology may be found in Benjamin Lewin, Genes VII, published by Oxford University Press, 2000, Kendrew et al. (eds.), The Encyclopedia of Molecular Biology, published by Blackwell Science Ltd., 1994; and Robert A. Meyers (ed.), Molecular Biology and Biotechnology: a Comprehensive Desk Top Reference, published by VCH Publishers, Inc., 1995 (ISBN 1 569-8).

As used herein “protein” means a polymer of amino acid residues linked together by peptide bonds. The term, as used herein, refers to proteins, polypeptides, and peptides of any size, structure, or function. Typically, however, a protein will be at least six amino acids long. Preferably, if the protein is a short peptide, it will be between 2 to 50 or more amino acid residues, or more preferably at least about 10 amino acid residues long. A protein may also be just a fragment of a naturally occurring protein or peptide. The term protein may also apply to amino acid polymers in which one or more amino acid residues is an artificial chemical analogue of a corresponding naturally occurring amino acid.

An amino acid polymer in which one or more amino acid residues is an “unnatural” amino acid, not corresponding to any naturally occurring amino acid, is also encompassed by the use of the term “protein” herein. A “fragment of a protein” means a protein which is a portion of another protein. For instance, fragments of a protein may be polypeptides obtained by digesting full-length protein isolated from cultured cells. A fragment of a protein will typically comprise at least six amino acids. More typically, the fragment will comprise at least ten amino acids. Preferably, the fragment comprises at least about thirty amino acids.

The present invention provides a filtration-based microarray system preferably using a nitrocellulose membrane as the filter substrate. The invention provides in one embodiment that multiple capture molecules are printed onto one or more nitrocellulose membrane filters to form microarrays. A mixture of multiple protein analytes in a liquid sample is filtrated through these membranes, and the analytes are consequently captured by their corresponding capture molecules during the filtration process. Although nitrocellulose membrane has been recognized as a high protein-binding capacity and hydrophilic material, and it has been commercialized to be substrates for protein chips with a two-dimensional surface adhered thereto, the present invention for the first time provides a highly efficient filtration-based hybridization assay. The invention demonstrates that the filtration-based protein chips essentially eliminate the diffusion limit that exists in the conventional protein chip design, leading to much faster hybridization kinetics, wider dynamic range, and more reliable and sensitive detection and quantification of analytes.

Cellulose membrane is the most commonly used protein-binding membrane [45]. The protein-binding affinity of a cellulose membrane can vary depending on the derivatives on its side chain (cellulose nitrate and cellulose acetate) and the surface charge. For most proteins, cellulose nitrate has 5-10 folds higher binding affinity than cellulose acetate; therefore, it has been widely used for immobilizing proteins [45][55], while cellulose acetate has been mostly used for bacteria isolation. Mixed cellulose ester, also know as nitrocellulose, is a mixture of both cellulose nitrate and cellulose acetate. For protein chip applications, nitrocellulose is believed to be better than other membranes because of its high protein binding capacity, hydrophilic surface and easiness of blocking non-specific binding [55].

Another commonly used protein-binding membrane is nylon. The advantage of nylon is that its surface chemistry can be easily modified. There have be different types of nylon membranes that appear to be positively charge, negatively charged or neutral. This leads to the versatility of nylon membrane to bind different types of molecules. Certain types of nylon can bind up to 400 microgram/cm² of proteins. However, this may lead to high non-specific protein binding which makes nylon membrane very difficult to block. The inventors tested MagnaCharge (Osmonics), and it consistently showed high background with three different blockers (BSA, Casein and Gelatin). However, other nylon membranes may perform well. For example, MagnaGraph is designed to avoid high background. These membranes can possibly be used for certain applications of filtration-based protein chip.

Another membrane that was tested by the inventors for filtration-based protein chip is UltraBind modified PES membrane. In this case, proteins are covalently linked to the aldehye activated PES surface. The results did not show a strong difference between hybridizations using nitrocellulose and those using Ultrabind. However, Ultrabind has a stronger surface scattering, and its surface is not as uniform as nitrocellulose. Also, unlike nitrocellulose, which can selectively bind unlabeled proteins, Ultrabind binds unlabeled proteins and fluorescentlly labeled protein equally well; therefore, they tend to have higher background after hybridization. The comparison of nitrocellulose to glass coverslide and other protein-binding porous media is shown in Table 1.

Both glass [1][58] and silicone wafer [10][53][54] require the modification of surface chemistry; however, compared with glass coverslide, glass fiber filter and silicone wafer can bind more proteins due to their porous feature. Silicone wafer, which is an array of closely packed microchannels as shown in FIG. 1b, has been used as the substrate for a flow-thru DNA chip (Genelogics) [8][49]. However, similar to glass coverslide, its hydrophobic surface compromises its potential in protein microarray applications. Further, the silanization of glass or silicone can be difficult to maintain a good batch-to-batch consistency.

TABLE 1
Comparison of different protein-binding substrates

| | | Chemical | Hydrophilic | |
|---|---|---|---|---|
| Material | Capacity (μg/cm²) | Modification | surface | Filtration Assay |
| Glass Coverslide | 1-10 | Yes | No | No (Not |
| (silanized) | | | | permeable) |
| Glass Fiber Filter | 10˜20 | Yes | No | Yes |
| (silanized) | | | | |
| Silicone wafer | Unknown | Yes | No | Yes |
| (silanized) | | | | |
| Nitrocellulose | 80˜150 | No | Yes | Yes |
| PVDF | ˜480 | No | No | Yes |
| Nylon | 170˜200 | Rarely | Yes | Yes |
| PAGE | Not Available | No | No | No (Too fragile) |

PVDF is also a membrane commonly used for immunoblotting assays. However, it is very hydrophobic and it is impractical to wet the membrane with organic solvents such as methanol during the printing process. Nylon has very high protein binding capacity, but the proteins are rather difficult to block. In addition to membranes, Polyacrylamide has been used to coat coverslide to immobilize antibodies, and the gel pad technique has been patented by Perkin-Elmer Bioscience. The problem with Polyarylamide gel pad is that it is very fragile and has to be supported by another material. Nitrocellulose filters used in the present invention have high protein-binding capacity and a very hydrophilic surface.

In addition to the difference in surface chemistry, nitrocellulose filter and silicone wafer are also fundamentally different in the pore morphology. Electron microscopy images in FIG. 1 demonstrate that, unlike silicon wafers comprising micro-channels perpendicular to the filter surface in FIG. 1b [35], nitrocellulose has a randomly oriented fiber network shown in FIG. 1a. In fact, nitrocellulose and silicon wafer are commonly referred to as depth filters and membrane filters, respectively. In general, a depth filter can withstand a high flow rate, and does not tend to be blocked by large particles, while membrane filters are mostly to capture larger filtrates and consequently easier to be blocked. In summary, nitrocellulose and silicone wafer are significantly different in their pore morphology, protein-binding chemistry, and surface hydrophobicity. The use of nitrocellulose membrane for filtration-based protein chips is also cost-effective.

Diffusion Limit in the Current Protein Chip Design [50]

As mentioned earlier, diffusion limit exists in solid-phase assays; it becomes more severe when the surface concentration of capture molecules is increased. To illustrate, consider a pair of molecules A and B that can react with each other to form a complex C

$$A + B {{⇔}_{{k}_{off}}}^{{k}_{o n}} C \qquad ( 1 )$$

where kₒₙ and k_{off} are the intrinsic on- and off-rate constants of the reaction. If molecule A is immobilized on a solid surface, with surface concentration Aₛ, then the reaction between A and B is

$${A}_{S} + {B}_{S} {{⇔}_{{k}_{off}}}^{{k}_{o n}} {C}_{S} \qquad ( 2 )$$

where B_{S} is the value of B at the surface (Note that Bₛ has the same dimension as B). The rate of production for C on the surface is

$$\frac{ⅆ {C}_{S}}{ⅆ t} = {k}_{o n} {A}_{S} {B}_{S} - {k}_{off} {C}_{S} \qquad ( 3 )$$

This also equals to the consumption of B at the surface. On the other hand, at steady state, the consumption of Bs at the surface is equal to the transport of B molecules to the surface. Therefore,

$$\frac{ⅆ {C}_{S}}{ⅆ t} = {k}_{o n} {A}_{S} {B}_{S} - {k}_{off} {C}_{S} = {k}_{m} ( {B}_{0} - {B}_{S} ) \qquad ( 4 )$$

where B₀ is the concentration of B in the bulk solution, and km is a lump parameter to describe the mass transport. Eliminating B_{S} from the above equation gives the production rate of C_{S} as

$$\frac{ⅆ {C}_{S}}{ⅆ t} = \frac{{k}_{m} {k}_{on}}{{k}_{m} + {k}_{on} {A}_{S}} {A}_{S} {B}_{0} - \frac{{k}_{m} {k}_{off}}{{k}_{m} + {k}_{on} A} {C}_{S} \qquad ( 5 )$$

When the concentration of B is low, As is approximately constant, and the surface concentration of C can be solved to be

$$\begin{matrix}{C}_{S} = \frac{{k}_{on}}{{k}_{off}} {A}_{S} {B}_{0} ( 1 - {ⅇ}^{- {k}_{r} t} ) \\ w h e r e & ( 6 ) \\ {k}_{r} = \frac{{k}_{off}}{1 + {A}_{S} {k}_{on} {k}_{m}^{- 1}} & ( 7 )\end{matrix}$$

From Equations 6-7, it is seen that the overall reaction kinetics is dependent on the intrinsic off-rates of the reaction, and the ratio between kₒₙA_{S} and kₘ. Note that kₘ has the dimension m/s. This indicates that, although increasing As can lead to larger values of C_{S}, i.e., higher signal intensity for protein chips, it also leads to a reduced value of kᵣ, and the reaction rate is slower. More specifically, when the hybridization is driven by shaking, the mass transport inside the boundary layer is primarily through diffusion, and km is related to the diffusivity of B and the thickness of boundary layer by kₘ=D/δ. For a typical protein molecule, the diffusivity D is around 5×10⁻⁷ cm²/s, and δ for a laminar flow on a plane having the scale of coverslide is roughly around 100 μm. Therefore, km is about 5×10⁻⁵ cm/s. If molecule A has a surface concentration of 15 μg/cm² on the surface, for antibodies whose molecular weight is 150,000 Dalton, this corresponds to A_{S}=100 nM-cm=1×10⁻⁷ M-cm. With a typical value of kₒₙ=10⁵ M/s for protein-protein interactions, kₒₙA_{S} is on the order of 1×10⁻² cm/s, which is three orders of magnitude higher than kₘ. Therefore, the apparent kᵣ is about 1000 times smaller than k_{off}, indicating that the diffusion limit is very significant. In practice, neither A_{S} nor B₀ may keep constant during the hybridization process, so the hybridization kinetics may be somewhat faster than what we predict here, but the diffusion limit still exists.

Filtration Assay can Eliminate the Diffusion Limit and Accelerate Hybridization Kinetics

In contrast to conventional protein chips with capture molecules immobilized on a solid surface, the filtration-based protein microarray is generated by printing spots on a protein-permeable substrate. Instead of relying on horizontal shaking, protein samples are filtrated through the protein chip. The flow filed perpendicular to the protein chip surface can give rise to much larger km values, thus significantly accelerate the hybridization rate. For example, if km=Vz=0.1 cm/s, then kr=0.67koff, which is fairly close to the intrinsic koff.

Multi-Chip Stacking of Filtration-Based Protein Chip System

The invention also provides a multi-chip stacking filtration assay. An apparatus is provided by the present invention of more than 1, 2, 3, 4, 5, 6, 7, 8, 9, or 10 or more protein microarrays stacked together, and the protein sample to be analyzed is filtrated through the entire stack. This apparatus and method requires a much smaller sample volume per chip, can significantly increase the throughout, and improve the hybridization consistency among multiple chips.

When the stacked chips contain different bound proteins, the stacked multichip system can selectively detect an analyte by using capture molecules printed on any layer of the stack. The same stacking approach can be used for a larger system, e.g., with 10 or more chips stacked together. For example, the invention contemplates a square filter with each side 22 mm in length, which is the same size as that of a coverslide. With a microarray of the present invention printed with the spot-to-spot spacing of 0.5 mm, then at least 1,600 spots can be arrayed on each chip. If 10 chips are used to form a stack, then 16,000 spots can be simultaneously hybridized. This is equivalent to at least 3 coverslide surfaces. If the spot size and spacing is reduced to 250 microns, then 64,000 samples can be screened simultaneously. Therefore, the stacking-hybridization system can significantly increase the throughput compared with the conventional microarray assay.

The stacking-filtration hybridization can also reduce the amount of sample needed for hybridization. It is always desirable to perform multiple tests with multiple chips to obtain more statistically reliable results, so there will be a compromise between the amount of sample per chip and the number of chips that can be tested. For prior art shaking assays, if the volume of sample used for each chip is below a certain limit, the hybridization will be completely dominated by slow diffusion, therefore the reaction kinetics will be extremely slow. However, if the stacking-filtration hybridization method of the present invention is used, although the sample volume per chip is the same, the total volume per test is still be large enough to generate a macroscopic convection flow through the stacked filters. Therefore, much better hybridization results can be obtained with the present invention.

Selection of Dye Molecules

The selection of dye molecules for labeling the analyte molecules in a sample influences the results of the microarray-based filtration assay. Directly labeling analyte proteins with fluorescent dyes is currently the dominant detection method [27]. Compared with enzymatic assays, detection through direct fluorescence labeling requires fewer steps and gives better spatial resolution. It is also superior to radioactive labeling, which has biohazard effects and intrinsic decay of signals.

A preferred fluorescent dye for protein chip application should possess a broad absorption spectrum so that it is readily excited by the lasers available in the commercial microarray scanner. Further, it should preferably have high extinction coefficient and quantum yield, high resistance to photobleaching and self-quenching. Because the white surface of nitrocellulose strongly scatters light and causes high background with 30-70 nm stokes shift, this dye molecule should also have long Stokes shift to reduce the scattering backgrounds. It has been reported that the intensity of scattering light decreases when the incident light wavelength becomes longer; therefore, long wavelength dyes including infrared fluorophores are very attractive in this application. Finally, the conjugation of the dye molecule to protein should be straightforward.

Given the above criterions, Alexa-647 has been identified as one preferred organic dye molecule for labeling proteins. Its excitation maximum is at 647 nm, compatible with our imaging instruments. Its extinction coefficient of 234,000/(M-cm) is among the highest of all organic dye molecules and is about 2 times higher than the commonly used Cy5 dye. Its quantum yield is also reasonably high and it has been reported that Alexa-647 conjugates are robust against self-quenching (Molecular Probes).

Although organic dyes have been widely used for DNA and protein microarray studies, they are not optimal since their extinction coefficients are relatively low, and photobleaching can become a problem. Further, their spectrum properties are less ideal. Recently, there have been intensive studies on potential substitutes for the conventional organic dye molecules; among other preferred candidates are phycobiliproteins and Quantum-dots.

Phycobiliproteins are a family of highly fluorescent proteins purified from Algae, and phycobilisomes are huge supra-molecular phycobiliprotein complexes [30][31][32]. Having a broad absorption spectrum, these fluorescent proteins are readily excited by laser. Further, their high extinction coefficients and quantum yield give rise to a much higher fluorescence intensity than conventional organic dyes. The supra-molecule PBXL-1 phycobilisome (Martek Bio) is a cluster of hundreds of phycobiliproteins. Containing 1400 chromophores, it is the brightest organic dye molecule in the market. Being 300 times brighter than FITC, it has been used to detect amol of proteins per band in western blotting [32]. Such high sensitivity is comparable to that of enzymatic assays. Moreover, PBXL-1 has a Stokes shift of up to 178 nm, which significantly reduces the scattering background. The detailed comparisons of phycobiliproteins and other dye molecules are shown in Table 2.

TABLE 2
The comparison of different dyes for fluorescence detection.

| | | | Molar | | | |
|---|---|---|---|---|---|---|
| | Excitation | Emission | Extinction | | | Molecular |
| | Wavelength | Wavelength | Coefficient | Quantum | Resistance to | Weight |
| Dye | (nm) | (nm) | (M⁻¹cm⁻¹) | Yield | Bleaching/Quenching | (Dalton) |
| B-phycoerythrin (BPE) | 546, 565 | 575 | 2.41 × 10⁶ | 0.98 | High/NA | 2.4 × 10⁵ |
| R-phycoerythrin (RPE) | 480, 546, 565 | 578 | 1.96 × 10⁶ | 0.82 | High/NA | 2.4 × 10⁵ |
| Cy3 | 550 | 570 | 130 000 | Not available | Low/Low | 1000 |
| Alexa 546 | 556 | 573 | 104,000 | Higher than | Intermediate/ | 1079 |
| | | | | Cy3 | Intermediate | |
| Quantum dots 560 | UV-550 | 560 | >10⁶ at 300 | Not available | High/High | ˜5 × 10⁵ |
| | | | nm | | | |
| PBXL1 phycobilisome | 480, 546, 565 | 660 | ˜10⁷ | Not available | High/NA | ˜10⁷ |
| Allophycocyanin (APC) | 650 | 660 | 700,000 | 0.68 | High/NA | 1.04 × 10⁵ |
| Cy5 | 649 | 670 | 250,000 | Not available | Low/Low | 1000 |
| Alexa 647 | 650 | 665 | 239,000 | Higher than | Intermediate/ | ˜1300 |
| | | | | Cy5 | Intermediate | |

Semiconductor nano-crystalline Quantum Dots (QDs) comprise another family of de novo dyes. Compared to conventional dye R6G [5][7][7], quantum dots 547 are 10 times brighter and 100 times more resistant to photobleaching. More important, their narrow emission peaks are virtually continuously tunable, with the broadest excitation spectrum compared to other dyes. In reality, a single 488 nm laser can excite all quantum dots with emission peaks from 500 nm and up. Using quantum dots, not only high detection sensitivity but also multicolor imaging can be readily achieved [6].

Although both phycobiliproteins and Quantum-dots have shown very promising applications in biological studies, their applications in microarrays technology have not heretofore been explored. In particular, to date quantum-dots have only been used in solution-phase assays. Therefore, in developing the filtration-based protein chips of the present invention, we have examined the possible applications of these de novo dyes.

### EXAMPLES

### Example 1

### Detection Specificity, Sensitivity, Kinetic Rate and Dynamic Range

In presenting some preferred features of the present invention, the following critical issues have been studied. The fundamentals of the protein-binding property of nitrocellulose, including the binding of both fluorescently labeled and unlabeled proteins. A comparison of nitrocellulose-based protein chips to glass coverslide based protein chips. A demonstration of the advantages of filtration-based protein chips compared to shaking based protein chips, including the specificity, accelerated kinetics, extended dynamic ranges, reduced background and reduced cross-reaction. The high throughput multi-chip stacking-hybridization system and its consistency and specificity. The application of phycobilisome and Quantum-dots as labeling agents in filtration-based protein chips. Performing sandwich assays using nitrocellulose filtration-based protein chips, and demonstrating its potential clinical applications.

### Materials and Methods

Materials

The primary nitrocellulose filters used have 0.8 micron pores in the cellulose membrane (containing primarily nitrocellulose and supported by a small portion of cellulose acetate) (Osmonics), with the maximal binding capacity of 140 μg/cm². For comparison purposes, two other types of filters were also tested. The first is a 0.45 micron GP-4 nitrocellulose membrane, and the second is 0.45 micron Ultrabind activated Polyethylenesulphone filter. Both of them were purchased from Pall Gelman.

The proteins used in this study include Human serum albumin (HSA), Bovine γ Globulin (BGG), neutravidin (Pierce), carcinoembryonic antigen (CEA) (US Biological), mouse IgG (MGG), protein G′ and streptavidin (Sigma), monoclonal mouse anti-HSA (AHSA1), monoclonal mouse anti-CEA (MACEA_H, MACEA_L) (Biospacific), polyclonal goat-anti-mouse IgG (Rockland), polyclonal mouse anti-CEA (PACEA), and mouse anti-HSA clone 11 (Sigma). Human plasma samples were kindly provided by Dr. Lily Yang at the Winship Cancer Institute of Emory University. Other reagents used include Tween-20 (Pierce), amino-modified glass coverslide (Coming) and dendrin filter holder (Pall Gelman).

Fluorescence labeling agents used include Alexa-488-SE, Alexa-546-SE, Alexa-647-SE (Molecular Probes), biotin-DNP-SE (Molecular Probe), and the phycobilisome dye molecule PB1L™ (Martek Bio Inc). Quantum-dots were kindly provided by Dr. Shuming Nie and Xiaohu Gao at the Department of Biomedical Engineering, Emory University.

Microarray Printing and Imaging

Printing of microarray on both nitrocellulose membrane and glass coverslide was performed using Biochip™ arrayer from Perking-Elmer Bioscience. It is a non-contact mode ink-jet arrayer that is capable of delivering accurate amount of protein samples. Although it has four tips that can spot four samples simultaneously in order to minimize the error introduced by tip-to-tip variance, in this study all the microarrays were produced using a single tip.

Prior to printing, the antibodies were dialyzed into spotting phosphate buffer that contains 0.05M of sodium phosphate monobasic and 0.05 M of sodium phosphate dibasic, pH 7.4. This buffer is recommended by the arrayer manufacturer and is different from the commonly used PBS, which contains 0.14M of sodium chloride. The reason is that high concentration of NaCl tends to crystallize inside the glass tip of the arrayer, whicht may influence the dispense of the sample. For short-term storage, the sample was stored under 4° C. If the antibodies were not used within a month, they would be aliquoted and stored under −20° C.

For different experiments, we have used two instruments to image the hybridized chips. The first is a Fuji imager. Although its resolution of 50 μm is less ideal for microarray studies, it is quite reliable in detecting fluorescence signals in 3-dimensional domains. Another instrument is the confocal-based array scanner (Perkin Elmer) that has a focal plane of about 30 μm thick. This implies that it cannot be used to compare different chips because slight variation of chip thickness can bias the signal intensity. However, due to its high resolution (from 50 μm down to 5 μm) and high sensitivity, we have used it to cross-check the results obtained using the Fuji imager.

Fluorescent Conjugation of Protein Molecules

The conjugation of proteins with dye or biotin molecules was through the primary amines on lysine groups of protein molecules. Different conjugation procedures were employed according to whether it is conventional organic dye molecules, Quantum-dots or Phycobilisome (protein conjugation prepared commercially by Martekbio).

The Alexa dyes and Biotin-DNP from Molecular Probes contain a highly amine-reactive succimidyl ester group; therefore, they readily react with the protein to be labeled. The conjugation procedure is straightforward [18][20]. Briefly, 5-6 molar excess of dye molecules were added to the concentrated protein sample, and reacted with stirring for 1.5 hours at room temperature. After reaction, the unreacted dye molecules were removed to eliminate background in the subsequent hybridization. Depending on the amount of protein sample, different methods were used. If the amount of proteins is less than 1 mg, which is true for most experiments, the unconjugated dyes were desalted by ultrafiltration using Microcon centrifugal filter to avoid further dilution and loss of sample. If the sample has more than 1 mg protein, conventional size exclusion chromatography was applied by using G-20 gel in PD-10 column (Amersham-Pharmacia). The labeled protein fractions after chromatography were concentrated by Microcon centrifugal filters if they were too dilute. To eliminate the nonspecific binding of protein sample onto the PES membrane in Microcon filter, 0.05% Tween was added to the protein sample prior to centrifugation.

Biotinylation is through the conjugation of Biotin-DNP-SE. Compared to Biotin-SE, this product contains a DNP moiety that has a specific absorbance at 362 nm. Conventionally, the degree of biotinylation is determined by the HABA assay, which is based on absorbance. For each HABA assay, it needs to consume 5˜10 μg of sample, and the results are not necessarily accurate and repeatable. As an alternative, using direct absorbance of DNP moiety is more accurate and the sample can be easily recovered. It was reported by the manufacturer that the DNP moiety does not significantly compromise the binding affinity between Biotin functional group and Biotin-binding proteins such as Streptavidin and Neutravidin. Using manual dot blotting followed by solid-phase hybridization, we also confirmed that the binding of fluorescently labeled Neutravidin to immobilized Biotin-DNP-Casein is similar to the binding of the Neutravidin to immobilized Biotin-DNP (data not shown).

Procedure of Filtration Assay and Shaking Assay

The current design of one embodiment of the present invention for a filtration-assay is shown in FIG. 3. Specifically, 13 mm filter holders were modified to form an open-face at one side to facilitate the loading of the sample. After the nitrocellulose protein chip is sandwiched in the holder, the other end of the holder is mounted to a syringe (BD scientific) via a luer connector. The syringe is then placed on a syringe pump and kept in vertical orientation. The sample can then be loaded to the top surface of the chip, and be hybridized with the chip by driving the syringe back and fourth. Using the current design of the device, about 100 μl of sample can be filtrated through the protein chip.

The procedures of both filtration assay and shaking assay are similar to common solid phase assays such as ELISA and western blotting. Briefly, the protein chip can be first blocked in 2% BSA before it is hybridized with the sample. For a filtration assay, the chip can be sandwiched in the filter holder as described above. For a shaking assay, the chip is placed into a pre-blocked well of 96-well microplate, which is then shaken on a regular circular shaker.

### Results

Fundamentals of Protein Binding Property of Nitrocellulose Membrane

The Wash-Off of Labeled Proteins from Nitrocellulose Filters.

To determine the influence of detergent and washing methods on wash-off of labeled proteins from nitrocellulose filter, BSA and BGG were conjugated with Alexa-647 as described above. A series of dilutions of the protein conjugates was spotted on a nitrocellulose membrane, and each sample was spotted 4 times, and the concentrations were given in Table 3.

TABLE 3
Samples used in testing the binding of labeled proteins

| | A | B |
|---|---|---|
| 1 | BSA-A647, 1.0 mg/ml (10 ng) | BSA-A647, 0.2 mg/ml (2 ng) |
| 2 | BSA-A647, 0.04 mg/ml (0.4 ng) | BSA-A647, 0.01 mg/ml |
| | | (100 pg) |
| 3 | BSA-A647, 0.002 mg/ml | |
| | (20 pg) | |
| 4 | | BGG-A647, 0.002 mg/ml |
| | | (20 pg) |
| 5 | BGG-A647, 0.01 mg/ml | BGG-A647, 0.04 mg/ml (0.4 ng) |
| | (100 pg) | |
| 6 | BGG-A647, 0.2 mg/ml (2 ng) | BGG-A647, 1.0 mg/ml (10 ng) |

To test the effects of Tween-20 concentrations, TBS, 0.05%-TTBS, 0.05%-TPBS and 0.2% TPBS were filtrated through the chips at 0.5 cm/s for 60 min, and the flourescence intensity of each spots were quantified. To compare the effects of different washing methods, 0.05% TTBS were washed under filtration at 0.1 cm/s, 0.5 cm/s and under shaking at 200 rpm for 60 min.

Washing-off of fluorescently labeled proteins from nitrocellulose may not represent that of unlabeled proteins since the hydrophobic moiety of fluorophores may change the surface chemistry of proteins, and thus the protein-nitrocellulose protein-Tween interactions. For a small amount of proteins immobilized on nitrocellulose membrane, it is difficult to directly test its extraction from membrane due to the low concentration of proteins in washed buffer. In this study, we examined the influence of Tween on the binding of unlabeled proteins by using an indirect method through antigen hybridization. The pattern of the chips for these assays is shown in FIG. 4. This array contains five different capture molecules, including one monoclonal antibody, two polyclonal antibodies, one protein other than antibody, and one protein-binding small molecule, and it covers different classes of protein-binding molecules. The filtration-based protein chips were assayed with a mixture of all the corresponding analytes. The concentrations of HSA, CEA, MGG and Neutravidin are 30 pM, 30 pM and 100 pM, respectively.

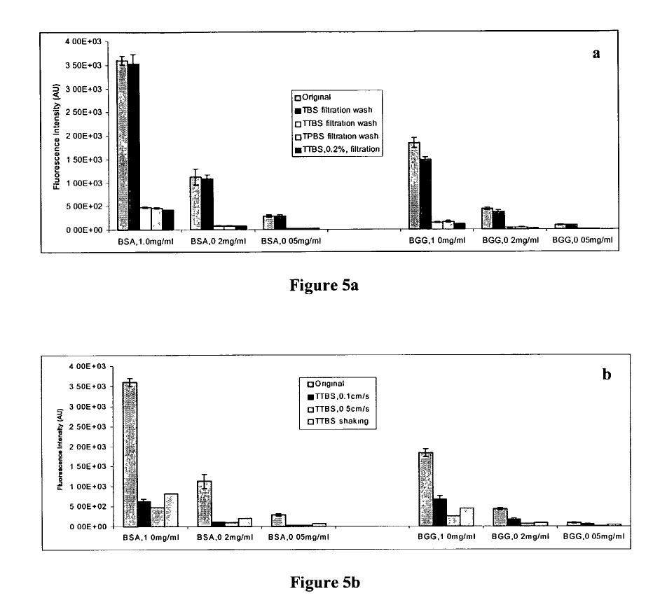

As demonstrated in FIG. 5a, with increased concentration of Tween-20 in the buffer, the wash-off of both fluorescently labeled proteins was increased. After 30 minutes washing, about 80% of BSA-A647 and 60% of BGG-A647 were washed away when 02% Tween was added.

FIG. 5b shows the effects of different washing methods. The results were similar for both proteins as well. In general, washing with filtration is more effective than with shaking, and increased filtration flow rate can further enhance the wash-off of labeled proteins.

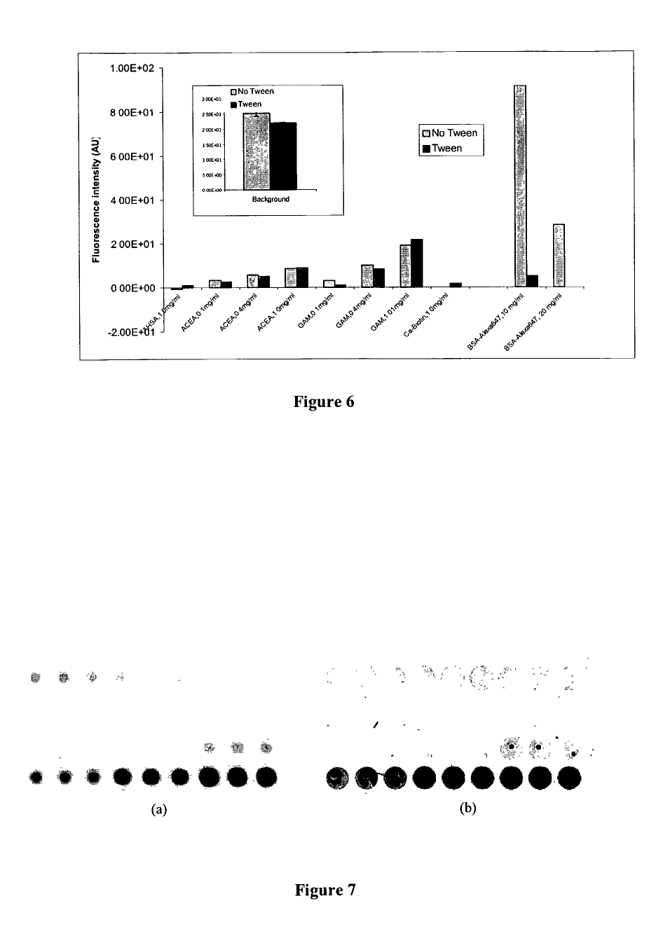

The wash-off of unlabeled proteins from the nitrocellulose surface was tested by detecting the fluorescently labeled analytes, as described in Methods section. Unexpectedly, the wash-off effects were completely different from the results for labeled proteins. In FIG. 6, it can be seen that including 0.2% Tween-20 in the hybridization buffer can reduce the fluorescent background on the chips (see insert in FIG. 6), and significantly reduced the fluorescent intensities of BSA-A647 spots; however, the signals of the spots due to hybridization are similar in both cases. Therefore, the Tween-20 cannot wash the unlabeled proteins off. Overall, using a low concentration of Tween-20 and giving moderate washing can reduce the background in hybridization and improve the signal contrasts.

Comparison of Nitrocellulose Protein Chips and Glass Coverslide Protein Chips

The array pattern is the same as that shown in FIG. 4. The coverslides were cut into square pieces that approximately 0.8 cm×0.8 cm after microarrays were printed onto them. Then the filter-chips were assayed under 0.4 cm/s filtration, and each coverslide chip was put into a well in a blocked 96-well microplate to hybridize under shaking at 200 rpm for 1 hour.

The spot qualities and hybridization results of nitrocellulose protein chips with protein microarrays printed on amine-modified glass coverslide were compared. The results were consistent with the previous results shown by S&S and an independent research using Fast™ slide. The nitrocellulose-based microarray showed better spot quality, better consistency, more reliable quantification, and higher sensitivity than the conventional coverslide-based microarray.

The spot quality on nitrocellulose filter is better than those on glass slide in terms of spot morphology and spot-to-spot variance. From FIG. 7, it can be seen that in contrast to the circular spots on nitrocellulose filter, the spots on coverslide appear to be less symmetric and non-uniform inside the spot. In particular, there were the nonhomogenous salt crystals inside the spots. The spot-to-spot variance on nitrocellulose is lower because the surface property is more uniform cross the chip than that of the modified coverslide. Also, the non-uniform absorption of proteins after printing may also contribute to the variance on coverslide.

Similarly, the nitrocellulose filter-based chips showed lower chip-to-chip variances as well. In addition to the reasons we mentioned for spot-to-spot variance, the non-uniform hybridizations under shaking may also contribute to the high chip-to-chip variances.

The spot intensities on nitrocellulose chip proportionally reflect the amount of dispensed antibodies, but on coverslides there is not a correlation between the spot intensities and the original antibody concentration. This is because the protein-binding capacity of modified glass surface is low, so the dispensed antibodies on the coverslide cannot proportionally bind to the proteins once the surface is saturated. Therefore, the coverslide chip may not have the accuracy necessary for protein expression profiling. Further, it will not be very effective to improve the sensitivity of an assay by deliver more antibodies to glass coverslides.

Since nitrocellulose surface can immobilize more proteins, it is most useful for detecting low-concentration samples. The actual sensitivity of an assay depends on the nature of the proteins involved, as well as the instruments to detect the signal. Since in the nitrocellulose membrane, the proteins are immobilized in a three-dimensional construct, it is not necessary to use confocal-array scanner to image. Confocal microscopy is necessary for coverslide imaging to reduce signal from other focus planes, however it may not be the best for imaging 3-D volumes such as nitrocellulose membranes.

We have compared the hybridization results of nitrocellulose-based microarrays and glass slide-based microarrays by using two types of instruments. When gel scanner was used, 60 pg/ml of CEA can be reliably detected by filtration-base protein chip, but even 1 ng/ml of CEA cannot be visualized on coverslide. This is consistent with a report by S&S indicating that certain antibodies can detect pg/ml of proteins on the nitrocellulose-coated slide. When the confocal-based array scanner was used, the two chips yield similar sensitivities.

Comparison of Filtration and Shaking Assays

An example of the microarray patterns is shown in FIG. 4. We first tested the specificity of the microarray by loading only one of the four analytes to the chip for each assay. We then compared the hybridization kinetics of filtration assay to shaking assay. As shown in FIG. 4, a mixture of HSA, CEA, MGG and NeutrAvidin (with 30 pM, 30 pM, 30 pM and 100 pM, concentration, respectively) were hybridized to the chips. To test dynamic ranges, a series dilution of mixed analytes was performed. The uniformity of hybridization across the surface of the chip was also tested.

The filtration-based protein chip significantly accelerates hybridization kinetics. Using the array design shown in FIG. 4, we have demonstrated that the filtration assay accelerates the hybridization not only when the capture molecules are antibodies, but also when the capture molecules are other proteins, small organic molecules. To illustrate the difference between the two types of assays, the fluorescent image of the arrays after 15 minutes and 45 minutes of hybridization are shown in FIG. 9. Note that although the signal of the BSA-A647 standards are similar in both assays, almost all the other spots show better signals in the filtration assay images. Clearly, for the two polyclonal antibodies, ACEA and GAM, the spots intensities were much higher in filtration-hybridization than in the shaking assay.

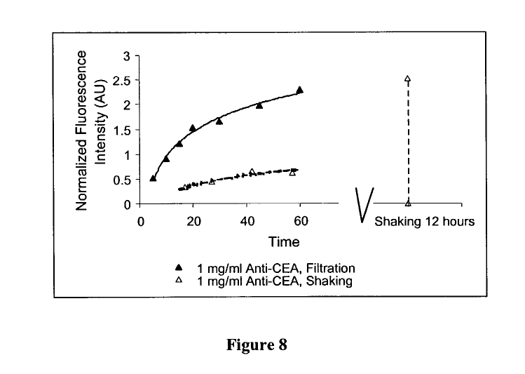

As an example, FIG. 8 compares the kinetics of hybridization of CEA to 1 mg/ml ACEA spots in both assays. After approximately an hour, the filtration assay was approaching equilibrium, but shaking assay only reached about 20% of the filtration assay intensity. After shaking overnight at room temperature, the shaking assay yielded a slightly higher signal than what filtration assay obtained within an hour.

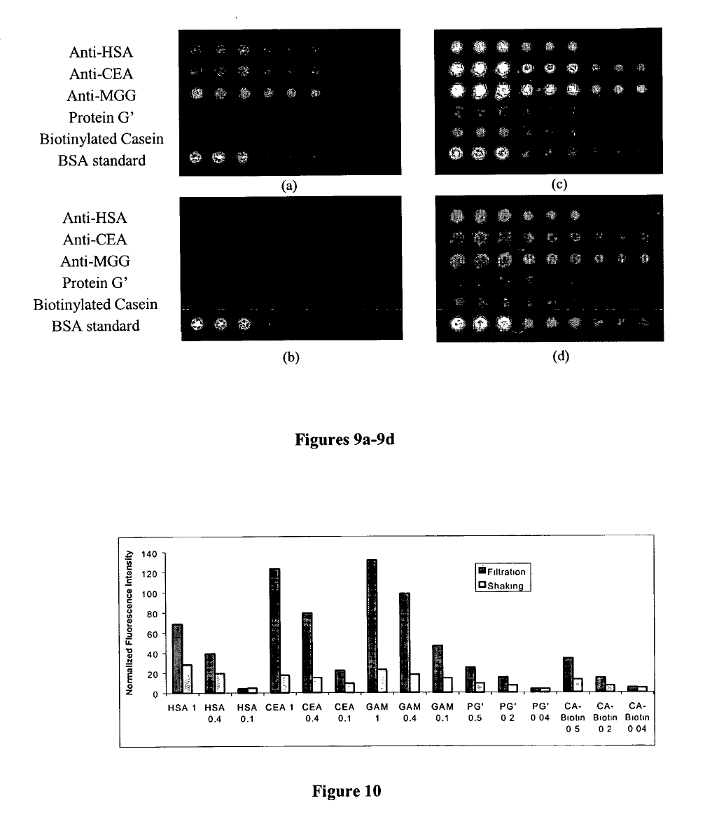

The quantification of all the spots are shown in FIG. 10. Note that the discrepancy between filtration and shaking assay is more prominent for spots with higher surface concentration. This shows that high surface concentration is more prone to diffusion limit, which agrees with the model prediction.

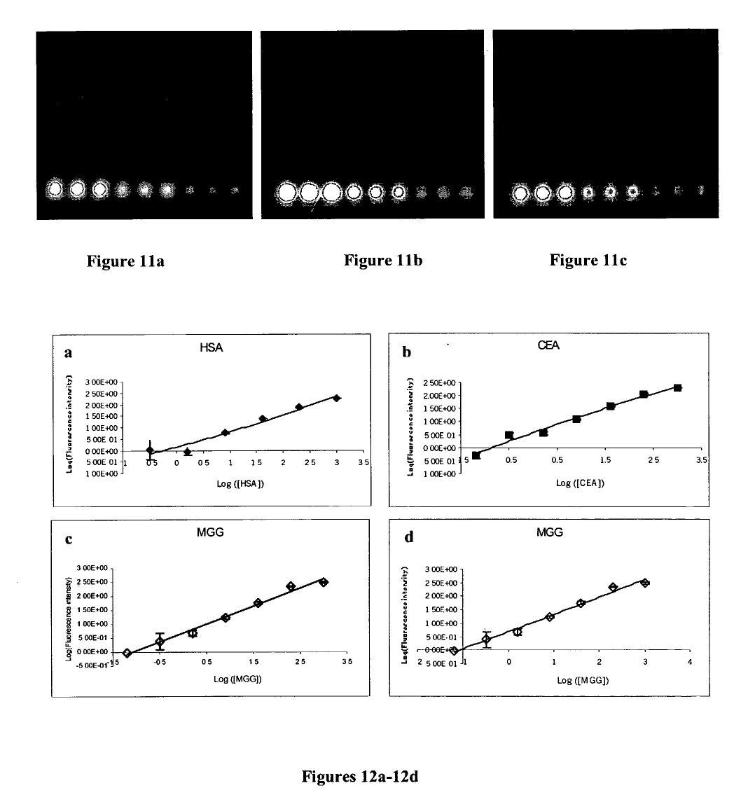

The filtration-based protein chip significantly accelerates hybridization kinetics. From FIG. 11, it is seen that within 30 min, the filtration-based protein chip was already able to detect multiple analytes with concentrations as low as 0.064 ng/ml.

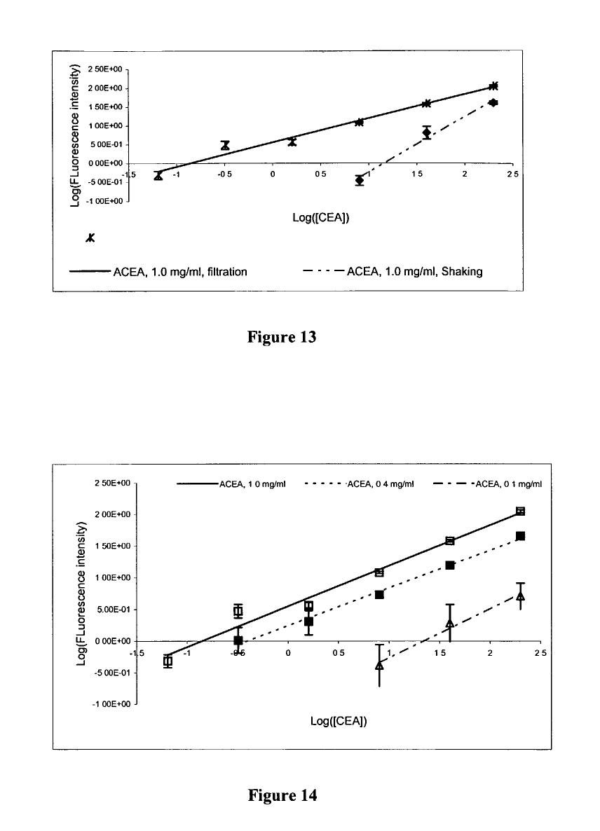

With the same reaction time, the filtration-based protein chips shown broader and linear dynamic ranges. Since the filtration-based protein chips can detect lower concentrations of proteins in a sample, the assay has a fairly linear dynamic range even within 30 minutes. The dynamic ranges for the four analytes are plotted in FIG. 12. It can be seen that Neutravidin has a slightly curved dynamic responses, but all other analytes appear to have a fairly linear response for more than three orders of magnitude. In contrast, due to diffusion limit, the shaking assay cannot detect the low concentration analytes efficiently within the same duration; therefore the shaking assay only has a very narrow dynamic range, slightly over 1 order, as shown in FIG. 13.

The error bars shown in FIG. 14 clearly indicate that the magnitude of standard deviation of the signal intensity decreases when the surface concentration of antibodies increases. This shows that higher antibody concentrations not only give a higher signal intensity but also lead to a better consistency in hybridization results.

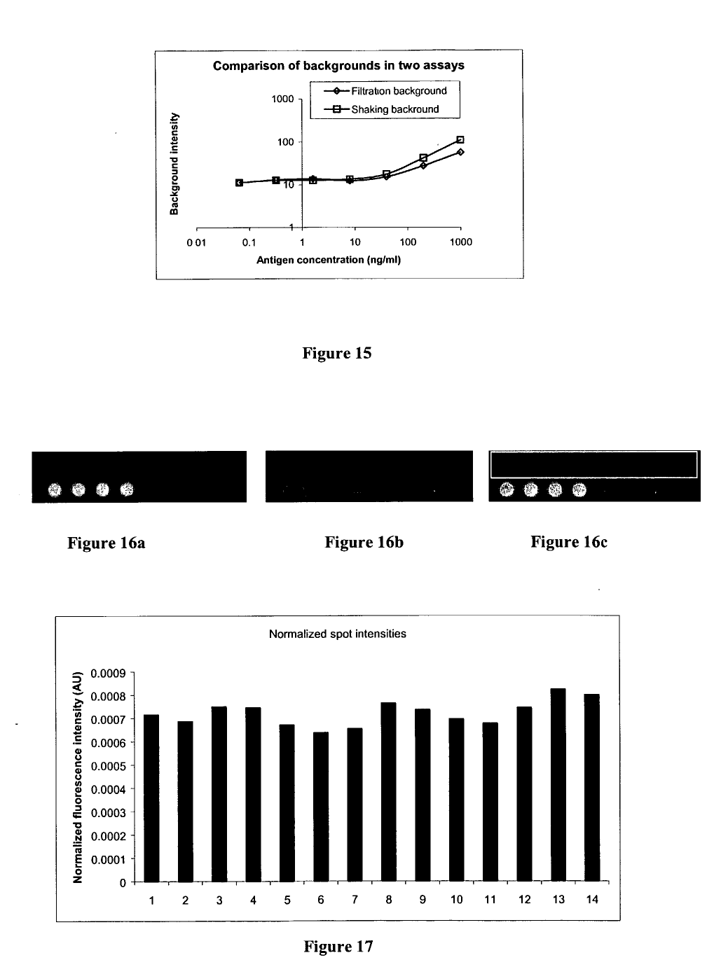

The filtration-based protein chips shows a reduction in background compared with shaking assays. Since nitrocellulose has the unique property that Tween buffer washes the labeled proteins more efficiently, better washing can improve the signal-to-noise ratio. As indicated above, in general filtration assay can wash off more non-specifically bound labeled proteins, thus reducing the background. FIG. 15 is a comparison of the background in assays using filtration and shaking. The direct consequence of the high background in shaking assay is a reduced signal-to-background ratio, i.e., the sensitivity of the assay. It is seen from FIG. 15 that the filtration assay also gives a clearer spot morphology than the shaking method.

The filtration-based protein chips shown reduction of cross-reaction and improved specificity. This was observed when the specificity of MGG binding to GAM was we tested. Although there was a very small amount of cross-reaction of MGG to AHSA and ACEA, the signal was not clearly visible for a short period of hybridization. However, after shaking overnight, even though the intensity of the GAM spots are comparable to those of the filtration assay after 60 min, the non-specific binding of MGG to AHSA and ACEA became much higher, as shown in FIG. 16.

Shown in FIGS. 17a & 17b are the results of filtration-hybridization of AHSA to HSA spots that were evenly printed along the diameter of a 13-mm diameter filter. Filtration rate was 0.1 cm/s. It can be seen that except at the very edge of the filter, the spot intensities are fairly consistent. Therefore, the filtration-based protein chips can provide uniform hybridization across the filter surface.

Multi-Stacked Filtration-Based Protein Microarray System

There is always a conflict between high throughput and the size of the spots. Reducing the spot size can increase throughput, but it also decreases the signal intensity. There is also a conflict between the number of chips required to obtain statistically reliable results and the amount of sample needed. For limited amount of sample, if multiple repeats of shaking-hybridization have to be done, the volume or concentration on each chip may become too small for efficient hybridization.

The present invention provides a stacked-filtration hybridizing assay to avoid the above conflicts. As illustrated in FIG. 2, a pile of nitrocellulose protein chips was stacked together, and the protein sample to be analyzed was filtrated through the filter stack. We have studied the following issues:

TABLE 4
Samples used in testing the binding of labeled proteins
in multi-chip stacking assay

| | A | B |
|---|---|---|
| 1 | AHSA, 1.0 mg/ml | ACEA, 1.0 mg/ml |
| 2 | GAM, 1.0 mg/ml | PG′, 1.0 mg/ml |
| 3 | CA-Biotin | BSA-A647, 1.0 μg/ml |
| 4 | BSA-A647, 2.0 μg/ml | BSA-A647, 5.0 μg/ml |

Two stacking-hybridization designs were tested, with 6 and 8 identical chips stacked, respectively. A total of 480 μl of the sample was tested, and the filtration volume was 300 μl. Filtration was at the flow rate of 0.5 cm/s. For comparison purposes, 4 chips were hybridized under shaking at 200 rpm. The reaction time for all the assays was 30 minutes. The top of the chip is defined in this example as the side at which the spots were dispensed.

Two types of arrays were studies, i.e., positive chips and negative chips. For the positive chips, spots 1A are AHSA, with concentration of 1.0 mg/ml, Spots 1B are AHSA, with concentration of 0.4 mg/ml. Spots 2A-6B are GAM, with concentration of 0.4 mg/ml. Spots 7 and 8 are BSA-A647, with concentration of 10 μg/ml and 2 μg/ml, respectively. The design of the negative chips is almost identical to that of the positive chips except that spots 1A and 1B are blank.

Two assays were performed with different number of chips. In the first assay, 5 chips were stacked together, with the middle one being a positive chip, all others were negative chips. In the second assay, one positive chip was sandwiched with 5 negative chips on each site. The sample contained 30 pM of HSA-A647 and 50 pM of MGG-A488. HSA and MGG were fluorescently labeled with two dyes that are well separated in their spectrum, so that one can simultaneously monitor the hybridization in two-color channels. As controls, single chip hybridization assays using filtration and shaking were also performed.

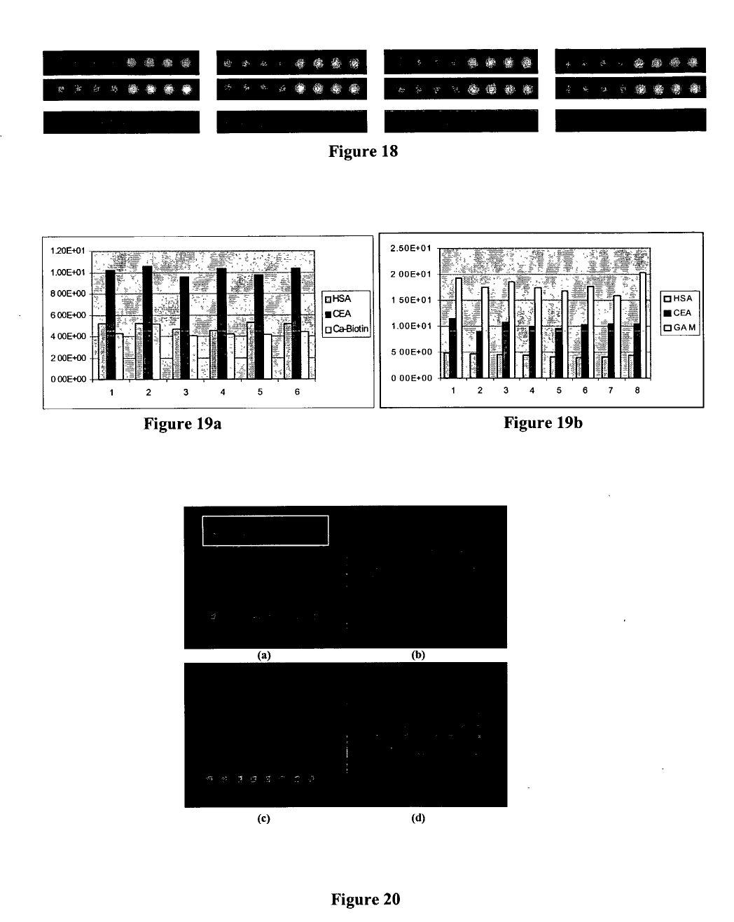

FIG. 18 illustrates the consistent hybridization of multi-analytes through the 8 protein chip layers (the top and middle rows) we tested. It can be seen that the fluorescence intensities are significantly higher than the 4 chips (the bottom row) hybridized under shaking although the volume per chip is the same. FIG. 19 shows quantitatively the fluorescence intensity of different analytes on different filters. It can be seen that chip-to-chip variations in the stacked hybridization assay are rather small. Further, the number on the x-axis represents the sequence of the filters from top to bottom, indicating the fluorescence intensity does not correlated with the location of filter.

When the stacked chips are different from each other, the stacked multichip system can selectively detect an analyte by using capture molecules printed on any layer of the stack. The images of the first chip (negative) and the 6^{th} chip (positive) were displayed in FIG. 20. Capturing of MGG-Alexa-488 was observed only on negative chips, and the intensity of the GAM spots was similar for the two chips. However, similar to the results obtained using an individual positive chip, the anti-HSA on chip 6 selectively captured the HAS-A647 in the sample, while the negative chip 1 did not show any signal of HSA. No cross contamination between adjacent chips was observed.

In this test we employed two dyes with different spectrum properties, and successfully visualized their signals in the two corresponding channels. This indicates that the filtration-based microarray chip can be used for assays using multi-channel detection.

From these results, it is expected that the same stacking approach can be used for a larger system, e.g., with 10 or more chips stacked together. For example, consider a square filter with each side 22 mm in length, which is the same as that of a coverslide. If a microarray is printed with the spot-to-spot spacing being 0.5 mm, then at least 1,600 spots can be arrayed on each chip. If 10 chips are used to form a stack, then 16,000 spots can be simultaneously hybridized. This is equivalent to the throughput of at least of 3 coverslide surfaces. If the spacing is reduced to 250 microns, then 64,000 samples can be screened simultaneously. Therefore, the stacking-hybridization system can significantly increase the throughput compared with the conventional microarray assay.

The stacking-filtration hybridization can also reduce the amount of sample needed for hybridization. It is always desirable to perform multiple tests with multiple chips to obtain more statistically reliable results, so there will be a compromise between the amount of sample per chip and the number of chips that can be tested. For prior art shaking assays, if the volume of sample used for each chip is below a certain limit, the hybridization will be completely dominated by slow diffusion, therefore the reaction kinetics will be extremely slow. However, if the stacking-filtration hybridization method of the present invention is used, although the sample volume per chip is the same, the total volume per test is still be large enough to generate a macroscopic convection flow through the stacked filters. Therefore, much better hybridization results can be obtained with the present invention.

Increasing Detection Sensitivity by Using Larger and Brighter Fluorescent Labeling Agents.

The following two major issues were studied: 1. Since both families of dyes have relatively large sizes and may slightly alter the surface chemistry of the conjugated proteins, will they have difficulty going through the nitrocellulose pores? 2. If they can go through the nitrocellulose pores, will filtration-assay facilitate their hybridization to the immobilized capture molecules?

Biotin and biotin-binding protein were used to address the above issues. Specifically, spots 1A, 1B, 2A and 2B contain 2 ng, 0.2 ng, 20 pg and 2 pg of BSA-Biotin-DNP, respectively. For phycobilisomes, streptavidin-conjugated PB1L was used, and streptavidin-A647 was used for comparison purposes. To have the same basis for comparison, the concentration of streptavidin-PB 1L and streptavidin-A647 were adjusted to 2 μg/ml and 20 ng/ml respectively so that the streptavidin molar concentrations were the same in both samples, which was around 300 pM. For the Quantum dot assays, Neutravidin were conjugated to QD-585 to form a NA-QD complex.

Neither imaging instruments used can provide the optimal excitations for PB1L and QD-585. Both 532 nm and 633 nm excitations were tested for PB1L. For QD-585, 488 nm and 532 nm excitations were used, but the optimal excitation should be around near-UV range such as 350 nm.

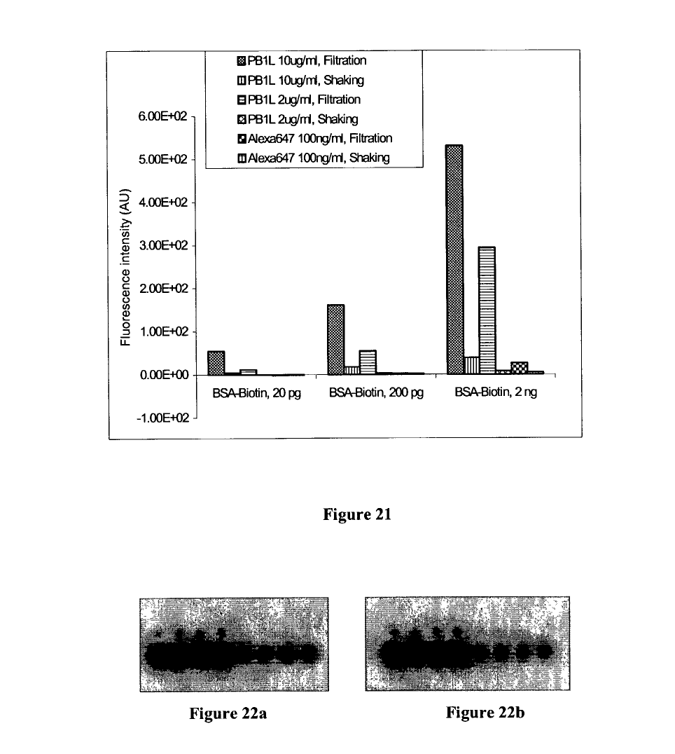

The results in FIG. 21 revealed the prominent differences between the filtration-PB1L detection and shaking-A647 detection. The excitation wavelength was 633 nm, which is between the excitation maximal of PB1L and A647. For the four types of test, the sensitivities ranking from high to low are: PB1L-filtration detection, PB1L-shaking detection, A647-filtration detection and A647-shaking detection. Especially, the PB1L-filtration detection showed the lowest detection limit of about 2 pg/spot on the surface, which is about 500 times mores sensitive than A647-shaking detection. We also used an excitation wavelength of 532 nm, and the intensity of A647 detection was significantly decreased while PB1L gave similar signals (Data not shown).

Phycobilisomes are considerably brighter than conventional organic dyes. The results in FIG. 21 indicate that, with the filtration-based assay, the diffusion limit caused by the slow motion of the large phycobilisome cluster can be eliminated.

We have also tested the compatibility of multi-chip stacking hybridization with PB1L detection. Two layers of nitrocellulose filters with BSA-Biotin spotted were stacked for hybridization, and they yielded fairly similar fluorescent signals, as shown in FIG. 22. This test further proves that PB1L can go through multi-layers of nitrocellulose filters without being trapped in the filters, which provides a new method that can improve the consistency of secondary detection and save expensive detecting reagents.

The filtration-based protein chips can also improve the hybridization of Quantum-dots conjugated proteins to surface immobilized capture molecules. BSA-Biotin-DNP spotted on nitrocellulose filters was detected by using 200 μl of NA-QD conjugates, which are diluted 200 folds from the original NA-QD mixture. The total NA concentration in the reagent is about 500 ng/ml. The excitation wavelength used was 488 nm.

Since the reaction molar ratio of NA to QD is much higher than 1, each QD particle could be multiply labeled by NA molecules, but the labeling efficiency was not estimated. The competition of the unlabeled NA molecules with the NA-QD complexes and the non-optimal excitation wavelength may have compromised the sensitivity of the current assay, but this preliminary study still clearly demonstrates the advantages of filtration-based protein chips in the detection assay using QD, and its ability to accelerate the surface hybridization of QD-conjugated proteins with immobilized capture molecules.

### Example 2

### Sandwich Assay Using Filtration-Based Protein Chips

The present invention demonstrates that compared to direct conjugation of analytes, the sandwich assay gives higher specificity and sensitivity. Further, the results of the filtration-based sandwich assay is much better than the shaking-based sandwich assay. We have demonstrated that combining the filtration-based microarrays technology of the present invention with a CEA two-site sandwich assay, 5 ng/ml of increased CEA concentration in blood plasma could be reliably detected.

### Methods

A demonstration of the capabilities of a protein chip designed to detect multiple cancer markers from a patient's blood was performed, wherein one of the markers is CEA [15]. The threshold of CEA concentration in blood is 5-10 ng/ml, which is about one millionth of the total protein concentration in human plasma. To selectively detect the changes of CEA level, the CEA antibody used has to have an association constant that is 6 orders lower for CEA than for other highly expressed proteins such as HSA and Human globulins. In the prior art this can be difficult, especially when proteins are directly conjugated with dye molecules, the hydrophobic moiety of dyes can increase the non-specific aggregation of proteins via hydrophobic interactions. To get around this problem, many clinical diagnosis assays employ two-antibody sandwich assays to improve the specificity of detection. Basically, a capture antibody is spotted on the chip first. After hybridization with unlabeled protein samples, a detection antibody is hybridized with the chip. This dual-antibody approach significantly improved the specificity of immunoassays.

Therefore, for this example, microarrays were designed for a sandwich assay. CEA was selected as the target molecule to be detected, and HSA as an internal control. The 6 groups of spots on this small-scale array are listed as follows:

| | |
|---|---|
| 1A: 1.0 mg/ml of ACEA_H; | 1B: 0.4 mg/ml of ACEA_H |
| 2A: 1.0 mg/ml of ACEA_L; | 2B: 1.0 mg/ml of PACEA; |
| 3A: 1.0 mg/ml of AHSA; | 3B: BSA-A647 standard |

The secondary detection antibodies for CEA and HSA were A647 conjugates of ACEA_L and AHSA-clone11, respectively. Their concentrations in the detections were 1.0 μg/ml and 1.5 μg/ml, respectively. The following three issues were studied:

(1) The sandwich assay verses direct labeling of analytes

(2) The detection of CEA elevation in human plasma sample, a model system

(3) The detection of CEA level human pancreatic cancer

In the first step of the above tests, the difference between filtration and shaking assays were compared. For the secondary detections, stacking the hybridized chips together after the first step and using stacking-hybridization can save significant amount of secondary detection reagents. However, a small amount of CEA dissociation and redistribution among different layers of chips can result.

### Results

10 ng/ml of CEA dissolved in a mixture of 50 μg/ml of HSA and 30 μg/ml of HGG was detected. This is roughly equivalent to 10 μg/ml of CEA in 80 mg/ml of total protein sample, which is similar to the total protein concentration in blood plasma. Both the CEA-positive and CEA-negative samples were directly conjugated with Alexa-488 before hybridization. Although the equivalent CEA concentration in the positive samples is already 1,000 fold higher than the range of interest, the shaking-based reaction still cannot reliably detect the difference between the CEA-positive and CEA-negative samples (data not shown).

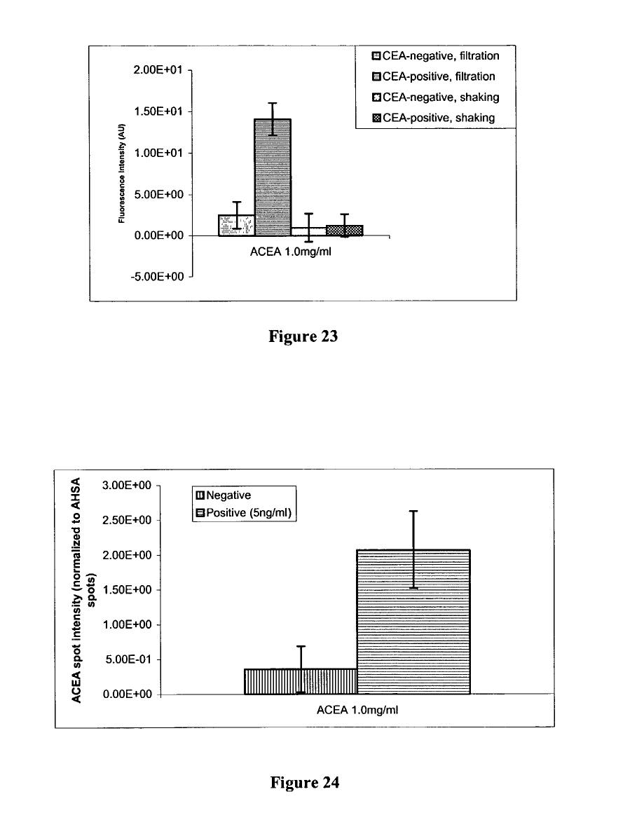

A secondary anti-CEA antibody was also conjugated with Alexa-A647. In the second step, four chips were stacked together. The results show that no matter which hybridization method was used in the first step, the sandwich assay shows improvements of false-positives over the direct detection, but the signal contrast is considerably better if the first step was conducted using filtration-based chips. Therefore, filtration-based assay can improve the capturing of CEA with the immobilized antibody as well as facilitate the subsequent secondary detection. FIG. 23 shows the quantification of these results.

Having shown the advantages of filtration-based sandwich assay over the direction detection method and shaking-based sandwich assay, we tested its capability of detecting small amount of CEA concentration changes in human plasma sample. 5 ng/ml of CEA was added to a healthy individual's plasma, and it was diluted 10 fold and subsequently hybridized with protein chips. As an internal control for comparing the CEA signals on different chips, the HSA concentration was also monitored by using a secondary anti-HSA (Sigma) antibody for each test. The results are compared to the original plasma sample (10 fold dilution). It clearly indicates that both reactions had similar hybridization efficiency since HSA signals appear similar for both chips, but the CEA-positive chip shows a large signal increase of ACEA spots, confirming that the filtration-based sandwich assay can reliably detect 5 ng/ml difference in CEA concentration, as shown in FIG. 24. The application of stacking-hybridization in the secondary detection did not significantly introduce CEA redistribution between the two chips since similar results were obtained when the secondary reaction were conducted under shaking.

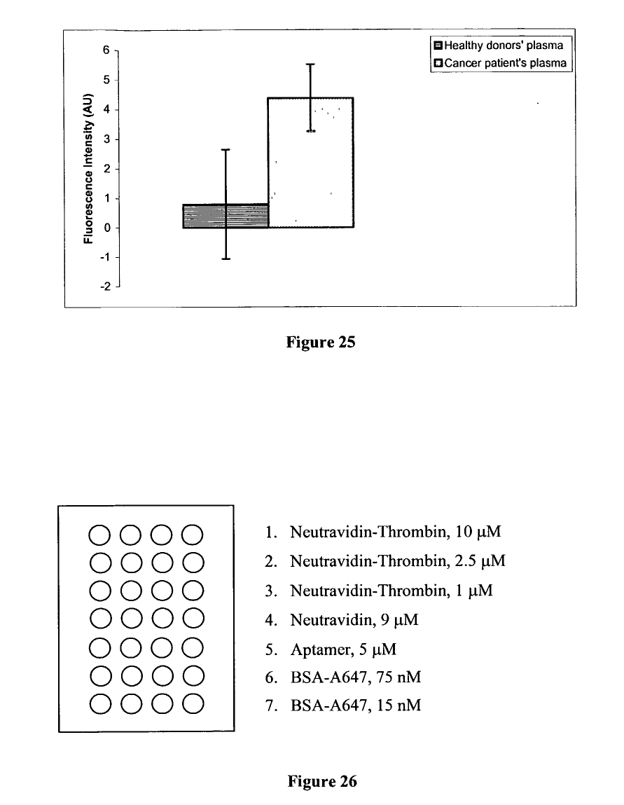

FIG. 25 shows the detection of CEA in pancreatic cancer patient's plasma. Negative controls were collected from two healthy donors. Two chips were hybridized with each negative control, while three tests were repeated for cancer plasma. Estimated by unpaired t-test, the results yielded from all the spots showed that the difference between this cancer sample and healthy donors' plasma is statistically significant.

### Example 3

### A Filtration-Assay Based Aptamer Chips for Thrombin Detection

Aptamer is a family of single strand oligonucleotide that is selected to have high specificity in binding to a class of molecules, including organic compounds, peptides and proteins [19]. Compared to antibodies, aptamers are easier to synthesize and more flexible for secondary detection. Therefore, many aptamers have been developed, including that for targeting thrombin, which has a relatively high affinity. [19][25]. In this study, we use thrombin-binding aptamer as a model system, and thrombin molecules conjugated with fluorescent dyes are detected by an aptamer microarray using both filtration and shaking assays. The specificity, sensitivity and dynamics range, and reaction kinetics of filtration-based assay wre compared with that of the shaking assay.

### Material and Method

Biotinylated thrombin-binding aptamers 5′-Biotin-C₆-GGTTGGTGTGGTTGG (IDT) was printed on nitrocellulose filters (NitroBind, Osmonics, with 13 mm in diameter and 0.45 pore size) to form an aptamer microarray. Since aptamer itself does not bind strongly to nitrocellulose membrane, and the binding can influence the conformation of apatomer, an indirect binding approach through neutravidin was used as a linker to facilitate indirect binding of aptomer to nitrocellulose. It is crucial to maintain the correct conformation of the aptamer for binding to thrombin, and the 6-carbon chain is believed to give the aptamer more freedom in folding into the correct conformation. The Circular Dichroism spectra of aptamer in buffer A (25 mM HEPES, 137 mM NaCl, 5 mM KCl, 1 mM CaCl₂, and 1 mM MgCl₂) was compared to the results of [48] to make sure that the aptamer molecules are folded correctly.

The neutravidin-aptamer complex was prepared by mixing an equal molar concentration (40 μM) of neutravidin and the biotinylated thrombin-binding aptamer for at least 30 min. The mixture was then diluted to desired concentration for being dispensed to the chip. Due to the high affinity between neutravidin and biotin (dissociation constant=10⁻¹⁵M), this reaction is believed to be very thorough. The mixture was printed to the nitrocellulose filter to form the microarray with a Biochip Arrayer (Packard Bioscience).

The design of the 7×4 microarray is shown in FIG. 26. Specifically, row 1 through row 3 are neutravidin-aptamer complexes, and row 4 and row 5 are pure neutravidin and pure aptamer respectively to provide the negative control for the test. Row 6 and row 7 are BSA conjugated with Alexa647 as fluorescent standard for the chip.

Human thrombin-α (Hameatologic Technologies Inc.) was labeled with Alexa-647 succinimidyl ester (A647) (Molecular Probes). Briefly, thrombin was dissolved in carbonate-bicarbonate buffer, pH 9.0, to 6 mg/ml. The A647 stock solution was then added to give a concentration of about 3 folds of the thrombin. After reaction for 1.5 hours at room temperature, the mixture was loaded to a Microcon centrifuge-filter (30,000 MWCO, Millipore) to remove excess dye molecules. The concentrations of the labeled thrombin and the conjugated A647 molecules were determined by absorbance spectra using the following formular:

C_{thrombin}=(A₂₈₀−0.03*A₆₅₀)/(ε_{Thrombin}*MW_{thrombin})(M), and C_{A647}=A₆₅₀/239000(M)

Where A₂₈₀ and A₆₅₀ are the absorbance of thrombin-A647 at 280 nm and 650 nm, respectively; ε_{Thrombin} is the extinction coefficient of thrombin at 280 nm, which is 1.83/(cm*mg/ml). MW_{thrombin} is the molecular weight of thrombin of 36700. The number of the dye molecules per thrombin is calculated by C_{A647}/C_{thrombin}

Human Serum Albumin (HSA, Pierce) was used as a negative control. The labeling of HSA and the determination of the labeling degree with A647 is similar to the procedure for thrombin except that ε_{HAS}=0.6/(cm*mg/ml), and MW_{BSA} is about 67000.

After the microarray was printed, the aptamer chips were blocked with blocking buffer (10 mg/ml BSA in buffer A) for 30 minutes with shaking, or for 10 minutes by pumping the blocking buffer through the aptamer chip. The thrombin-A647 sample was diluted in washing buffer (1 mg/ml BSA in buffer A) to the desired concentration. For the filtration assay, the aptamer chips were loaded to a 13 mm-filter acetal copolymer holder, and 200 μl of thrombin-A647 was driven through the filter back and forth by a syringe pump (Kd Scientific) with a flow rate of 2 ml/min. For shaking assay, the chips were placed into a well of a 96-well plate. The well was pre-blocked for 30 minutes to prevent nonspecific binding of thrombin to its wall. The plate was shaken at 200 rpm. For kinetics studies, different reaction periods were selected to generate the kinetics curve. For other studies with filtration-based aptamer chip, 10 minutes reaction time was used.

After reaction, the chips were washed for 20 seconds in washing buffer to remove non-specific binding. They were then imaged using either a Fuji gel imager or a ScanArray 4000 array scanner (Packard Biosciences). A control chip without reacting with the sample was also imaged simultaneously, and the fluorescence intensity on each hybridized chip was normalized by the fluorescence standards on the control chip with the NIH Imaging software. This was to avoid the random errors due to the variations of the instrument conditions, and to facilitate comparison between different images with varied brightness and contrast.

### Results

In labeling thrombin and HAS, the labeling efficiency of A647 to proteins are strongly dependent on the availability of exposed lysine residues, therefore it can vary from protein to protein. The labeling ratio of thrombin and HSA with Alexa-647 was determined to be 0.9 and 4.0, respectively, by using the procedure defined in the previous section.

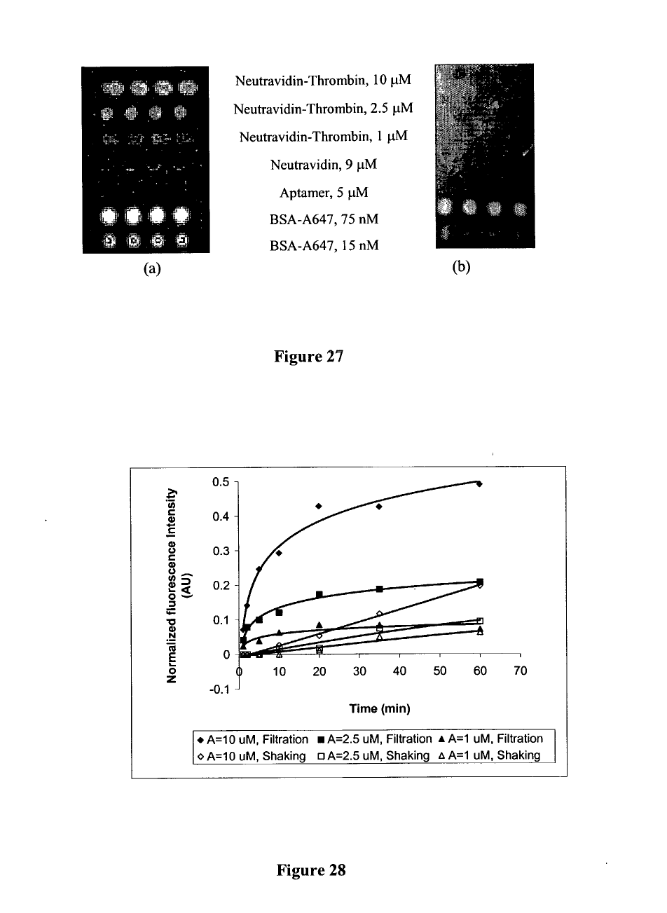

To demonstrate the specificity of the aptamer chip in thrombin detection, 0.5 mg/ml of pure neutravidin was spotted the microarray as a negative control. After performing the filtration assay for 10 minutes, it was found that neutravidin only weakly interacted with thrombin-A647, as shown in FIG. 27a. It was estimated that the surface concentration of neutravidin was about 10 times of that of the neutravidin-aptamer in spots in the 3^{rd} row in FIG. 26, but compared with the neutravidin-aptamer complex, neutravidin bound only about 20% of thrombin, and this background can be significantly reduced by using 0.05% tween-20 in the sample, indicating that the binding was due to nonspecific hydrophobic interactions between neutravidin and thrombin. It was also found that aptamer alone cannot detect thrombin in the sample since it could be washed away easily from the chip. We also tested HSA-A647 as a negative control, and found that neutravidin-aptamer did not cross-react with HSA. This confirms that the interaction between aptamer and thrombin is very specific (see FIG. 27b).

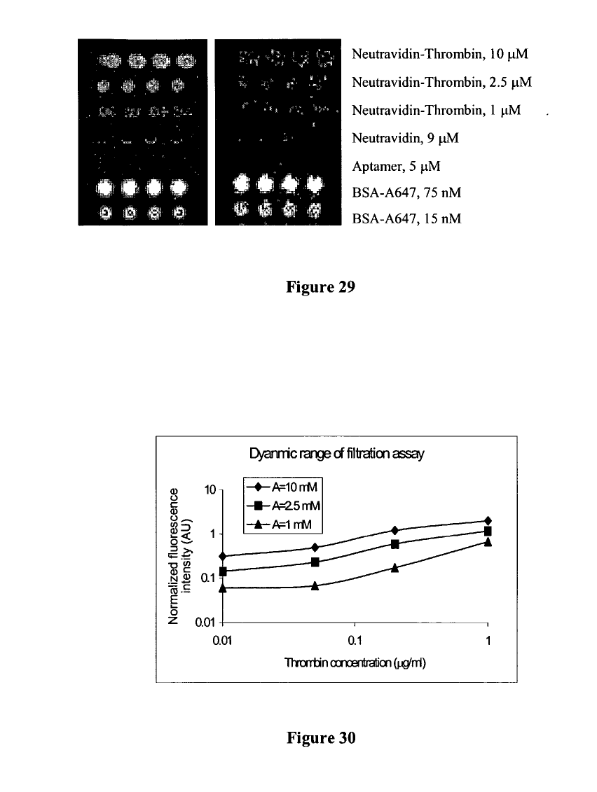

The reaction kinetics of filtration assay and shaking assay are compared for 10 ng/ml of thrombin-A647, and the normalized fluorescence intensities as a function of time are shown in FIG. 28. The signal of aptamer chips after reacting 10 minutes are compared with that of the shaking assay in FIG. 29. It can be seen that for any of the three Neutravidin-Aptamer concentrations, the kinetics of filtration assay is much faster than that of shaking assay. The signal ratios between filtration assay and shaking assay at 20 and 60 minutes are tabulated in Table 5. Clearly, to achieve high detection sensitivity, more aptamer molecules need to be immobilized on the surface. However in this case the diffusion limit is more severe. When thrombin concentration is low, the diffusion limit in a shaking assay is also more sever owing to the depletion of thrombin molecules inside the boundary layer.

TABLE 5
Ratios of signal obtained in filtration assay and shaking assay

| Aptamer Concentration | 20 min | 60 min |
|---|---|---|
| A = 10 μM | 6.5 | 2.5 |
| A = 2.5 μM | 4.7 | 2.1 |
| A = 1 μM | 3.3 | 1.3 |

To determine that dynamic range of the assay, a series of thrombin-A647 samples with concentrations ranging from 10 ng/ml to 1 μg/ml were pumped through the aptamer-chip in alternative directions for 10 minutes, and the signals of the chips were imaged by the Fuji gel imager. The fluorescence intensity as a function of thrombin concentration after normalization with fluorescent standards is shown in FIG. 30. It can be seen that the curves are linear over two orders of magnitude, except that with apatomer concentration of 1 μM and thrombin concentration of 0.01 μg/ml, the signal is higher than expected. This deviation could be due to the unreliable performance of the assay at low signal level. Overall, the dynamic range of the assay spans from the lowest detection limit to at least 1 μg/ml. The correlation between the signal and thrombin concentrations higher than 1 μg/ml was not studies since it is of little interest in high-sensitivity assays.

### REFERENCES

[1] Aebersold R H, Teplow D B, Hood L E, Kent S B Electroblotting onto activated glass. High efficiency preparation of proteins from analytical sodium dodecyl sulfate-polyacrylamide gels for direct sequence analysis. J Biol Chem. 1986 Mar 25;261(9):4229-38.

[2] Anderson N L, Anderson N G. Proteome and proteomics: new technologies, new concepts, and new words. Electrophoresis. 1998 Aug;19(11):1853-61.

[3] Black D L Protein diversity from alternative splicing: A challenge for bioinformatics Cell 2000, 103:367-370.

[4] Borrebaeck C A Antibodies in diagnostics—from immunoassays to protein chips. Immunol Today. 2000 Aug;21(8):379-82.

[5] Bruchez M. Jr Moronne, M., et. al., Semiconductor Nanocrystals as fluorescent biological labels, Science, 281(25):2013-2015.

[6] Chan W C, Maxwell D J, Gao X, Bailey R E, Han M and Nie S Luminescent quantum dots for multiplexed biological detection and imaging Curr Opin Biotechnol. 2002 Feb;13(1):40-6. Review.

[7] Chan W C, Nie S Quantum dot bioconjugates for ultrasensitive nonisotopic detection. Science. 1998 Sep 25;281(5385):2016-8.

[8] Cheek B J, Steel A B, Torres M P, Yu Y Y, Yang H Chemiluminescence detection for hybridization assays on the flow-thru chip, a three-dimensional microchannel biochip. Anal Chem 2001 Dec 15;73(24):5777-83

[9] Clark C R, Kresl J J, Hines K K, Anderson B E. An immunofiltration apparatus for accelerating the visualization of antigen on membrane supports. Anal Biochem. 1995 Jul 1;228(2):232-7.

[10] Claudine E. Williams, Olivier Theodoly, Thomas T. Waigh Adsorption of a Highly Charged Polyclectrolyte on Hydrophobic Surfaces. (Physique de la Matiere Condensee, College de France and CNRS, Paris, France)

[11] Dabbousi, B. O.; et. al. (CdSe)ZnS Core-shell quantum dots: Synthesis and characterization of a size series of highly luminescent nanocrystallites J. Phy. Chem. B 1997, 101:9463-9475.

[12] Davies C et. al. The immunoassay handbook edited by Wild D 1994 M Stockton press.

[13] Dutt M J, Lee J H Proteomic analysis. Curr Opin Biotechnol. 2000 Apr;11(2):176-9. Review.

[14] Feng H P A protein microarray Nature structural biology 7(10):829.

[15] Gold P, Goldenberg N. A. The Carcinoembryonic Antigen (CEA): Past, Present, and Future http://www.mjm.mcgill.ca/issues/v03n01/cea.html Portions of this review have been published in Perspectives in Colon and Rectal Surgery 9(2);

[16] Green L S, Bell C, Janjic N. Aptamers as reagents for high-throughput screening. Biotechniques. 2001 May;30(5):1094-6, 1098, 1100 passim.

[17] Gygi S, Rochon Y, Franza B, Aebersold R Coorelation between protein and mRNA abuance in yeast. Mol Cell Bio 1999, 19:1720-1730.

[18] Haugland R P, Handbook of Fluorescent probes and research chemicals, seventh edition Molecular probes, 1999

[19] Hermann T, Patel D J. Adaptive recognition by nucleic acid aptamers. Science. 2000 Feb 4;287(5454):820-5.

[20] Hermanson G T Bioconjugate techniques Academic Press, INC 1996

[21] Horton J K, James J E, Swinburne S, O'Sullivan M J. Novel and rapid immunofiltration assays with enhanced chemiluminescence detection. J Immunol Methods. 1990 Dec 31;135(1-2):289-91.

[22] IJsselmuiden O E, Herbrink P, Meddens M J, Tank B, Stolz E, Van Eijk R V. Optimizing the solid-phase immunofiltration assay. A rapid alternative to immunoassays. J Immunol Methods. 1989 Apr 21;119(1):35-43.

[23] Jain K K. Applications of proteomics in oncology. Pharmacogenomics. 2000 Nov;1(4):385-93. Review

[24] Jain K K. Applications of proteomics in oncology. Pharmacogenomics. 2000 Nov;1(4):385-93. Review

[25] Lee M, Walt D R. A fiber-optic microarray biosensor using aptamers as receptors. Anal Biochem. 2000 Jun 15;282(1):142-6.

[26] Luo L Y, Diamandis E P. Preliminary examination of time-resolved fluorometry for protein array applications. Luminescence. 2000 Nov-Dec;15(6):409-13.

[27] MacBeath G, Schreiber S L Printing proteins as microarrays for high-throughput function determination. Science. 2000 Sep 8;289(5485):1760-3

[28] MacBeath G, Schreiber SL Printing proteins as microarrays for high-throughput function determination. Science. 2000 Sep 8;289(5485):1760-3

[29] Madoz-Gurpide J, Wang H, Misek D E, Brichory F, Hanash S M. Protein based microarrays: a tool for probing the proteome of cancer cells and tissues. Proteomics. 2001

[30] Martek Biosciences corporation Conjugation Protocols using PBXL markers http://www.martekbio.com/fluor/MTK8.pdf

[31] Martek Biosciences corporation Conjugation protocols using phycobiliproteins http://www.martekbio.com/fluor/MTK2.pdf

[32] Martek Biosciences corporation Phycobilisome technical description http://www.martekbio.com/fluor/MTK6.pdf

[33] Mendoza L G, McQuary P, Mongan A, Gangadharan R, Brignac S, Eggers M High-throughput microarray-based enzyme-linked immunosorbent assay (ELISA). Biotechniques. 1999 Oct;27(4):778-80, 782-6, 788.

[34] Morais S, Gonzalez-Martinez M A, Abad A, Montoya A, Maquieira A, Puchades R. A comparative study by the enzyme-linked immunofiltration assay of solid phases used in the development of flow immunosensors. J Immunol Methods. 1997 Oct 13;208(1):75-83.

[35] Muller, F. Birner A. and Osele U. G. Structuring of Macroporous Silicon for Applications as Photonic Crystals. Journal of Porous Materials 7, 201-204 (2000).

[36] Pandey A, Mann M Proteomics to study genes and genomes, Nature, 405(6788):837-846

[37] Pinon J M, Puygauthier-Toubas D, Lepan H, Boulant J, Marx-Chemla C, Dupont H. Enzyme-linked immuno-filtration assay applied to accelerated immunodetection of gel transferred proteins on immobilizing matrices. J Immunol Methods. 1990 Jul 20; 131(1):143-5.

[38] Pinon J M, Puygauthier-Toubas D, Lepan H, Marx C, Bonhomme A, Boulant J, Geers R, Dupont H. Rapid detection of proteins by enzyme-linked immunofiltration assay after transfer onto nitrocellulose membranes. Electrophoresis. 1990 Jan;11(1):41-5.

[39] Reen D. J. Enzyme-linked immunosorbent assay (ELISA) Chapter 47 in “Basic protein and peptide protocols”, edited by Walker J. M. 1994, Humana press

[40] Roda A, Guardigli M, Russo C, Pasini P, Baraldini M Protein microdeposition using a conventional ink-jet printer. Biotechniques. 2000 Mar;28(3):492-6

[41] Rye P D, Nustad K. Immunomagnetic DNA aptamer assay. Biotechniques. 2001 Feb;30(2):290-2, 294-5.

[42] Service R F, Protein arrays step out of DNA's shadow, Science, Sep, 2000, 289:1673

[43] Sheikh S H, Abela B A, Mulchandani A Development of a fluorescence immunoassay for measurement of paclitaxel in human plasma Anal Biochem. 2000 Jul 15;283(1):33-8.

[44] Shi, L., DNA Microarray (Genome Chip)—monitoring the Genome on a Chip www. gene-chips.com

[45] Shields M J, Siegel J N, Clark C R, Hines K K, Potempa L A, Gewurz H, Anderson B. An appraisal of polystyrene-(ELISA) and nitrocellulose-based (ELIFA) enzyme immunoassay systems using monoclonal antibodies reactive toward antigenically distinct forms of human C-reactive protein. J Immunol Methods. 1991 Aug 9;141(2):253-61.

[46] Silzel J W, Cercek B, Dodson C, Tsay T, Obremski RJ Mass-sensing, multianalyte microarray immunoassay with imaging detection. Clin Chem. 1998 Sep;44(9):2036-43.

[47] Sklar L A Real-time spectroscopic analysis of ligand-receptor dynamics. Annu Rev Biophys Biophys Chem 1987;16:479-506.

[48] Smirnov I, Shafer R H Effect of loop sequence and size on DNA aptamer stability. Biochemistry. 2000 Feb 15;39(6):1462-8.

[49] Steel A, Torres M, Yang H J, et al. The flow-thru chip™: a three dimensional biochip platform Microarray Biochip Technology, edited by Mark Schenma 2000

[50] Stenberg M, Nygren H. Kinetics of antigen-antibody reactions at solid-liquid interfaces. J Immunol Methods 1988 Oct 4;113(1):3-15

[51] Stenberg M, Nygren H. Kinetics of antigen-antibody reactions at solid-liquid interfaces. J Immunol Methods.

[52] Stenberg M, Nygren H. A diffusion limited reaction theory for a solid-phase immunoassay. J Theor Biol. 1985 Apr 7;113(3):589-97.

[53] Stephan Heyse, Horst Vogel, Michael Sänger, and Hans Sigrist. Covalent. attachment of functionalized lipid bilayers to planar waveguides for measuring protein binding to biomimetic membranes.

[54] Susan C., et. Al, Protein adhesion on SAM coated semiconductor wafers: hydrophobic versus hydrophilic surfaces Sandia report, 2000 Sand2000-3016

[55] Tonkinson J L, Stillman B A Nitrocellulose: a tried and true polymer finds utility as a post-genomic substrate. Front Biosci 2002 Jan 1 ;7:c1-12

[56] Velculescu V E, Zhang L, Vogelstein B, and Kinzler K W Serial Analysis Of Gene Expression. 1995, Science 270, 484-487

[57] Walter G, Bussow K, Cahill D, Lueking A and Lehrach H Protein arrays for gene expression and molecular interaction screening Current Opinion in Microbiology 2000, 3:298-302.

[58] Weetakk H H Preparation of immobilized proteins covalently coupled through silane coupling agents to inorganic supports. Appl Biochem Biotechnol. 1993 Jun;41(3):157-88.

[59] Werthen M, Nygren H Effect of antibody affinity on the isotherm of antibody binding to surface-immobilized antigen. J Immunol Methods. 1988 Nov 25;115(1):71-8.

[60] Zhu H, Klemic J F, Chang S, Bertone P, Casamayor A, Klemic K G, Smith D, Gerstein M, Reed M A, Snyder M Analysis of yeast protein kinases using protein chips Nat Genet. 2000 Nov;26(3):283-9.

[61] Zubritsky E., Spotting a microarray system (product review) Analytical chemsitry 2000, 72(23):761A-767A.

Throughout this application, various publications are referenced. The disclosures of all of these publications and those references cited within those publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and Examples be considered as exemplary only. Those skilled in the art will recognize, or will be able to ascertain using no more than routine experimentation, many equivalents to the specific embodiments of the invention described herein. Such equivalents are intended to be encompassed by the following claims.

We claim:

1. A filtration-based microarray chip comprising,
a. a random oriented microporous filtration substrate having a planar aspect and comprising charged cellulose esters, and
b. a plurality of different analyte-specific capture molecules attached to the substrate in a microarray, wherein the filtration-based microarray chip permits a fluid solution to flow therethrough and at least a portion of analytes to be captured thereto.

2. The microarray chip of claim 1, wherein the charged cellulose esters are cellulose nitrate, cellulose actetate or mixtures thereof.

3. The microarray chip of claim 1, wherein the charged cellulose esters are nitrocellulose.

4. The microarray chip of claim 1, wherein the filtration substrate micropores are between about 0.05 and 10 microns in diameter.

5. The microarray chip of claim 1, wherein the analyte is a protein.

6. The microarray chip of claim 1, wherein the analyte is an antibody.

7. The microarray chip of claim 1, wherein the analyte is obtained from a sample of bodily fluid.

8. The microarray chip of claim 1, wherein the analyte is obtained from a sample of cell lysate.

9. The microarray chip of claim 1, comprising more than ten different analyte-specific capture molecules attached to the substrate in a microarray.

10. The microarray chip of claim 1, wherein the analyte-specific capture molecules are proteins.

11. The microarray chip of claim 1, wherein the analyte-specific capture molecules are antibodies.

12. The microarray chip of claim 1, wherein the analyte-specific capture molecules are apatomers.

13. An apparatus for analyte detection comprising a plurality of filtration-based microarray chips of any of claims 1-12 aligned with planar aspects in parallel such that a solution flows through the plurality of chips.

14. The apparatus of claim 13, further comprising a holder for the chips and a means for washing a solution of analytes repeatedly through the plurality of chips.

15. The apparatus of claim 14, wherein the means for washing a solution of analytes repeatedly through the plurality of microarray chips is a syringe in fluid communication with a chamber housing the chip holder and the aligned chips.

16. A method of detecting a subject analyte, comprising combining the micrarray chip of claim 1 with a sample suspected of containing the subject analyte, and detecting capture of the analyte on the substrate to determine the presence of the subject protein in the sample.

17. The method of claim 16, wherein the detection of the subject analyte indicates the presence of a disease marker in the sample.

18. The method of claim 16, wherein the analyte is obtained from a sample of bodily fluid.

19. The method of claim 16, wherein the analyte is obtained from a sample of cell lysate.

20. The method of claim 16, wherein detection of the analyte is achieved by labeling the analyte before capture.

21. The method of claim 16, wherein the analyte is labeled with a fluorescent dye.

22. The method of claim 21, wherein detection of the analyte is achieved by labeling the analyte after capture with a labeled antibody.

23. The method of claim 21, wherein the antibody is labeled with a fluorescent dye.

24. The method of claim 16, wherein the detection is performed with single- or multiple-photon microscopy, or time-resolved fluorescence microscopy.
